(19) United States
(12) Patent Application Publication
(10) Pub. No.: US 2009/0170745 A1
(43) Pub. Date: Jul. 2, 2009

(54) SUBTILISIN FROM BACILLUS PUMILUS AND DETERGENT AND CLEANING AGENTS CONTAINING SAID NOVEL SUBTILISIN

(75) Inventors: Marion Merkel, Koeln (DE); Petra Siegert, Haan (DE); Susanne Wieland, Dormagen-Zons (DE); Karl-Heinz Maurer, Erkrath (DE); Cornelius Bessler, Duesseldorf (DE)

Correspondence Address: Ratner Prestia, P.O. Box 980, Valley Forge, PA, 19482, US

(73) Assignee: Henkel AG & Co. KGaA, Duesseldorf (DE)

(21) Appl. No.: 12/268,702

(22) Filed: Nov. 11, 2008

Related U.S. Application Data
(63) Continuation of application No. PCT/EP2007/003998, filed on May 7, 2007.

(30) Foreign Application Priority Data
May 11, 2006 (DE) 10 2006 022 224.5

(51) Int. Cl. C11D 7/42; C12N 15/57; C12N 15/75; C12N 9/54; C12S 11/00; C12S 9/00; C12N 15/77; C12N 15/70; C12N 1/21

(52) U.S. Cl. 510/320; 510/392; 536/23.2; 435/320.1; 435/221; 435/252.31; 435/252.32; 435/252.33; 435/252.3

(57) ABSTRACT

A novel subtilisin-type alkaline protease from Bacillus pumilus and sufficiently related proteins and derivatives thereof. Also washing and cleaning agents comprising the novel subtilisin-type alkaline protease, sufficiently related proteins and derivatives thereof, corresponding washing and cleaning methods, and washing and cleaning agents and other possible technical uses.

### CROSS REFERENCE TO RELATED APPLICATIONS

This application is a continuation of PCT Application Serial No. PCT/EP2007/003998, filed May 7, 2007, and claims priority to German Patent Application Serial No. 102006022224.5, filed May 11, 2006. These applications are incorporated by reference herein, in their entirety and for all purposes.

### BACKGROUND

The present invention relates to a novel alkaline protease of the subtilisin type from Bacillus pumilus and adequately related proteins and their derivatives. It also relates to detergents and cleaning agents having this novel alkaline protease of the subtilisin type, adequately related proteins and their derivatives, corresponding washing and cleaning methods and use thereof in detergents and cleaning agents as well as other possible technical uses.

Enzymes are established active ingredients of detergents and cleaning agents. Proteases induce degradation of protein-based soiling on the items to be cleaned, such as textiles or hard surfaces. At best there are synergistic effects between the enzymes and the other components of the respective agents. The development of detergent proteases is based on naturally formed enzymes, preferably formed microbially. Such enzymes are optimized by essentially known mutagenesis methods, e.g., point mutagenesis, deletion, insertion or fusion with other proteins or protein parts or via other modifications, for use in detergents and cleaning agents. Of the detergent and cleaning agent proteases, subtilisins assume an excellent position because of their favorable enzymatic properties, such as stability or optimum pH.

Proteases of the subtilisin type (subtilases, subtilopeptidases, EC 3.4.21.62), in particular subtilisins, are classified as serine proteases based on catalytically active amino acids. They are formed and secreted naturally by microorganisms, in particular by Bacillus species. They act as nonspecific endopeptidases, i.e., they hydrolyze any acid amide linkages, which are in the interior of peptides or proteins. Their optimum pH is usually in the definitely alkaline range. A review of this family can be found, for example, in the article “Subtilases: Subtilisin-like proteases” by R. Siezen, pages 75-95 in “Subtilisin Enzymes,” edited by R. Bott and C. Betzel, New York, 1996. Subtilisins are suitable for a number of possible industrial applications, as ingredients of cosmetics and in particular as active ingredients of detergents or cleaning agents.

The most important subtilisins and the most important strategies for their further industrial development are listed below.

The subtilisin BPN′, which originates from Bacillus amyloliquefaciens and/or B. subtilis, is known from the articles by Vasantha et al. (1984) in J. Bacteriol., vol. 159, pp. 811-819 and by J. A. Wells et al. (1983) in Nucleic Acids Research, vol. 11, pp. 7911-7925. Subtilisin BPN′ serves as a reference enzyme of subtilisins, in particular with regard to the numbering of the positions.

The protease subtilisin Carlsberg is presented in the publications by E. L. Smith et al. (1968) in J. Biol. Chem., vol. 243, pp. 2184-2191 and by Jacobs et al. (1985) in Nucl. Acids. Res., vol. 13, pp. 8913-8926. It is naturally formed by Bacillus licheniformis and is available under the brand name Maxatase® from the company Genencor International, Inc., Rochester, N.Y., USA and under the brand name Alcalase® from the company Novozymes A/S, Bagsvaerd, Denmark.

Subtilisins 147 and 309 are distributed by the company Novozymes under the brand names Esperase® and/or Savinase®. They are originally obtained from Bacillus strains disclosed in the patent application GB 1243784.

Subtilisin DY was originally described by Nedkov et al., 1985 in Biol. Chem. Hoppe-Seyler, vol. 366, pp. 421-430.

Additional proteases of the subtilisin type that have been isolated from the Bacillus strains are described in the more recent patent applications WO03/054184 and WO03/054185.

### BRIEF DESCRIPTION OF THE DRAWINGS

FIG. 1 shows an alignment of the amino acid sequences of the inventive protease from Bacillus pumilus with the most similar known subtilisins, each in the mature form, i.e., the processed form. The numbers stand for the following proteases: 1. Inventive protease from Bacillus pumilus (SEQ ID NO:3); 2. Protease Q5XPN0 from Bacillus pumilus (Swiss-Prot) (SEQ ID NO:4); 3. Protease Q6SIX5 from Bacillus pumilus (Swiss-Prot) (SEQ ID NO:5); 4. Protease Q9 KWR4 from Bacillus pumilus (Swiss-Prot) (SEQ ID NO:6); 5. Protease Q2HXI3 from Bacillus pumilus (Swiss-Prot) (SEQ ID NO:7).

FIG. 2 shows the expression vector pAWA22 derived from pBC16, having a promoter from B. licheniformis (PromPLi) and downstream from that a Bcl I restriction splice site (cf. Example 2 and Bernhard et al. (1978), J. Bacteriol. 133 (2), pp. 879-903).

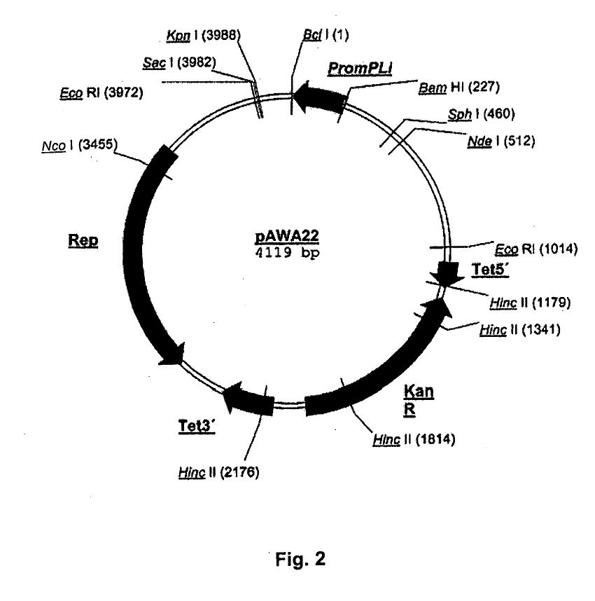

### DETAILED DESCRIPTION

A strategy to improve the washing performance of subtilisins consists of substituting randomly or in a targeted manner individual amino acids in the known molecules with others and testing the resulting variants for their contributions to washing performance. The enzymes may also be improved with regard to their allergenicity with certain amino acid exchanges or deletions.

To improve the washing performance of subtilisins, the strategy of insertion of additional amino acids into the active loops has been pursued. This strategy should be applicable in principle to all subtilisins belonging to one of the subgroups I-S1 (true subtilisins) or I-S2 (highly alkaline subtilisins).

Another strategy to improve performance consists of altering the surface charges and/or the isoelectric point of the molecules and thereby altering their interactions with the substrate. Furthermore, point mutants having a reduced pH-dependent variation in molecular charge have also been described. A method of identifying variants that are said to be suitable for use in detergents and cleaning agents has also been derived from this principle; in this method, all the variants disclosed have at least one exchange in position 103. In general, variants with one exchange in position 103 are described often in the literature, optionally in combination with a number of other possible exchanges. An alternative possibility for improving performance in detergents and cleaning agents consists of increasing the hydrophobicity of the molecules, which can have an influence on the stability of the enzyme.

Another method for modulating the efficiency of proteases consists of forming fusion proteins. For example, fusion proteins of proteases and an inhibitor such as the Streptomyces subtilisin inhibitor have been described in the literature. Another possibility is, for example, coupling to the cellulose binding domains (CBD) derived from cellulases to increase the concentration of active enzyme in the immediate vicinity of the substrate or coupling of a peptide linker and then polymers thereto to reduce allergenicity and/or immunogenicity.

Methods of creating random amino acid exchanges may be based on the phage display, for example. A modern direction in enzyme development consists of combining elements of known related proteins by random methods to form novel enzymes having properties not previously achieved. Such methods are also combined under the umbrella term “recombination.” This includes the following methods, for example: the StEP method (Zhao et al. (1998), Nat. Biotechnol., vol. 16, pp. 258-261), random priming recombination (Shao et al. (1998), Nucleic Acids Res., vol. 26, pp. 681-683), DNA shuffling (W. P. C. Stemmer (1994), Nature, vol. 370, pp. 389-391) or recursive sequence recombination (RSR; WO 98/27230, WO 97/20078, WO 95/22625) or the RACHITT method (Coco, W. M. et al. (2001), Nat. Biotechnol., vol. 19, pp. 354-359). A review of such methods is also given in the article “Directed evolution and biocatalysis” by Powell et al. (2001), Angew. Chem., vol. 113, pp. 4068-4080.

Another strategy, in particular a supplementary strategy, consists of increasing the stability of the respective proteases and thus increasing their efficacy. Stabilization by coupling to a polymer has been described for proteases used in cosmetics, for example; better skin tolerance has been achieved in this way. However, stabilizations by point mutations are more common for detergents and cleaning agents in particular. Thus proteases, for example, may also be stabilized with regard to use at elevated temperatures in particular by exchanging certain tyrosine residues with other amino acid residues. Other possibilities that have been described for stabilization by point mutagenesis include, for example:

Exchange of certain amino acid residues with proline;

Introduction of more polar or more charged groups on the surface of the molecule;

Increasing the binding of metal ions, in particular by mutagenesis of the calcium binding sites;

Blockage of autolysis by modification or mutagenesis;

Ascertaining the positions relevant for stabilization by analysis of the three-dimensional structure.

It is known that proteases may be used together with α-amylases and other detergent enzymes, in particular lipases, to improve the washing performance and/or the cleaning performance. Likewise, those skilled in the art are familiar with the use of proteases in detergents in combination with other active ingredients, such as bleaching agents or soil-release agents.

Furthermore, it is known that some proteases that have become established for use in detergents are also suitable for cosmetic purposes or for organochemical synthesis.

The various technical fields of use presented here require proteases having different properties, with regard to the reaction conditions, stability or substrates specificity, for example. Conversely, the possible industrial applications for proteases, e.g., in the context of a detergent formulation or cleaning agent recipe, depend on other factors such as the stability of the enzyme with respect to high temperatures, with respect to oxidizing agents, denaturing thereof by surfactants, on folding effects or on desired synergisms with other ingredients.

There is thus still a high demand for proteases that can be used industrially and cover a broad spectrum of properties up to and including very subtle differences in performance because of the variety of areas of use.

The basis for this has been expanded through novel proteases which may in turn be developed further in a targeted manner with regard to special areas for use.

The object of the present invention was thus to discover another as yet unknown protease. The wild-type enzyme should preferably be characterized in that it at least approximates the enzymes established for this purpose when used in a corresponding agent. The contribution toward the performance of a detergent or cleaning agent was of particular interest here.

Other objects of the present invention may be regarded as providing proteases, in particular those of the subtilisin type, which have an improved stability in comparison with the prior art with respect to temperature influences, fluctuations in pH, denaturing agents or oxidizing agents, proteolytic degradation, high temperatures, acidic or alkaline conditions or with respect to a change in redox ratios. Additional objects might be seen in a reduced immunogenicity and/or a reduced allergenic effect.

Another particular object of the present invention was to discover proteases that have a good washing performance at temperatures of 20° C. to 60° C., preferably an improved washing performance in comparison with the proteases disclosed in the prior art, in particular those of the subtilisin type.

Other partial objects consisted of making available nucleic acids that code for such proteases and making available vectors, host cells and production methods that may be utilized to produce such proteases. Furthermore, corresponding agents, in particular detergents and cleaning agents, corresponding washing and cleaning methods and corresponding possible applications for such proteases should be made available. Finally, possible technical applications for the proteases thereby discovered should be defined.

This object is achieved by alkaline proteases of the subtilisin type having amino acid sequences at least 98.5% identical to the amino acid sequence given in the sequence protocol under SEQ ID NO. 2 from positions 109 through 383 and/or deviate in at most four amino acid positions with respect to this amino acid sequence.

Even more preferred are those having a greater measure of identity with the novel alkaline protease from Bacillus pumilus, i.e., those which deviate in only two or three amino acid positions, preferably in only one amino acid position, most especially preferably being the alkaline protease from Bacillus pumilus itself.

Additional approaches to achieving the object and/or achieving the partial objects and thus separate subject matters of the invention consist of nucleic acids whose sequences are sufficiently similar to the nucleotide sequence defined in SEQ ID NO. 1 or that code for inventive proteases, in corresponding vectors, cells and/or host cells and manufacturing methods. Furthermore, corresponding agents, in particular detergents and cleaning agents, corresponding washing and cleaning methods and corresponding possible applications for such proteases are made available. Finally, possible technical applications are defined for the proteases thereby discovered.

The use of alkaline proteases from Bacillus pumilus in detergents and cleaning agents is already known to those skilled in the art. For example, in EP0572992 the use of alkaline proteases from Bacillus pumilus in detergents and cleaning agents is described. The protein sequence of the enzymes described there is not given.

The publications by Pan et al. (Current Microbiology 49 (2004), 165-169), Aoyama et al. (Microbiol. Immunol. 44(5) (2000), 389-393), Huang et al. (Current Microbiology 46 (2003, 169-173), Aoyama et al. (Appl. Microbiol. Biotechnol. 53 (2000), 390-395), Yasuda et al. (Appl. Microbiol. Biotechnol. 51 (1999), 474-479) and Miyaji et al. (Letters in Applied Microbiology 42 (2006), 242-247) are to be regarded as the most proximate prior art for the subject matter of the present invention. These documents describe the use of Bacillus pumilus proteases having the sequences Q6SIX5 (Swiss-Prot), Q9 KWR4 (Swiss-Prot), Q5XPN0 (Swiss-Prot) and/or Q2HXI3 (Swiss-Prot), which have a very high homology with the inventive protease. Depilation of leather has been described as a possible application for some of these proteases because these proteases are largely inactive with respect to collagen and therefore can be used for depilation of leather without damaging the leather in the treatment. Enzymatic treatment of soymilk and soymilk products is mentioned as an additional possible application. Degradation of zein, the main constituent of the protein in the seed of corn, has been mentioned as a possible application for the protease Q2HXI3. However, use of these enzymes in detergents and cleaning agents is not disclosed in these documents.

The naturally formed alkaline protease of the subtilisin type on which the present invention is based, as can be concluded on the basis of the examples, is available from the culture supernatant of a novel Bacillus pumilus strain that has been identified as such by the DSMZ (Deutsche Sammlung für Mikroorganismen und Zellkulturen [German Collection of Microorganisms and Cell Cultures]). For the purpose of repeatability, in accordance with the Budapest Treaty, a plasmid containing the nucleic acid sequence of the inventive enzyme was deposited with the DSMZ (German Collection of Microorganisms and Cell Cultures, Braunschweig) with the deposit number DSMZ 18097.

The present patent application has pursued the strategy of discovering from a natural habitat a protease-forming microorganism and thus a naturally formed enzyme that meets the requirements stipulated as thoroughly as possible.

As described in the examples of the present patent application, such an enzyme has been discovered in the alkaline protease from Bacillus pumilus.

As can be ascertained beyond the biochemical characterization performed by the German Collection of Microorganisms and Cell Cultures, this strain secrete a proteolytic activity. According to SDS polyacrylamide gel electrophoresis, it has a molecular weight of 27 kD with an isoelectric point of more than 8.5, as determined according to isoelectric focusing.

The nucleotide sequence of the novel inventive alkaline protease from Bacillus pumilus is defined in the sequence protocol of the present patent application under SEQ ID NO. 1. It comprises 1152 bp. The amino acid sequence derived from it is given in SEQ ID NO. 2. It comprises 383 amino acids, followed by a stop codon. Of this, the first 108 amino acids are presumably not contained in the mature protein, so this presumably yields a length of 275 amino acids for the mature protein.

These sequences were compared with the protease sequences obtainable from the generally accessible databases Swiss-Prot (Geneva Bioinformatics (GeneBio) S.A., Geneva, Switzerland; http://www.genebio.com/sprot.html) and GenBank (National Center for Biotechnology Information NCBI, National Institutes of Health, Bethesda, Md., USA) to ascertain the proteins having the greatest homology.

The measure of homology is a percentage of identity, which can be determined, for example, according to the method given by D. 3. Lipman and W. R. Pearson in Science 227 (1985), p. 1435-1441. This value may refer to the entire protein or to the respective region to be assigned. Similarity, another homology term, also includes preserved variations, i.e., amino acids having a similar chemical activity, in the consideration, because they usually have chemical activities within the protein. In the case of nucleic acids, only the percentage identity is known.

At the DNA level, the following genes have been identified as the most similar for the entire gene: (1) sequence Q5XPN0 from Bacillus pumilus (Swiss-Prot) with 94% identity, (2) sequence Q6SIX5 from Bacillus pumilus (Swiss-Prot) with 91% identity, (3) sequence Q9 KWR4 from Bacillus pumilus (Swiss-Prot) with 91% identity, (4) sequence Q2HXI3 from Bacillus pumilus (Swiss-Prot) with 91% identity.

At the level of the DNA coding for the mature protein: (1) sequence Q5XPN0 from Bacillus pumilus (Swiss-Prot) with 95% identity, (2) sequence Q2HXI3 from Bacillus pumilus (Swiss-Prot) with 91% identity, (3) sequence Q6SIX5 from Bacillus pumilus (Swiss-Prot) with 90% identity, (4) sequence Q9 KWR4 from Bacillus pumilus (Swiss-Prot) with 90% identity.

At the level of amino acids for the entire preproprotein, the most similar have been identified as follows: (1) sequence Q2HXI3 from Bacillus pumilus (Swiss-Prot) with 98% identity and/or deviations in seven amino acid positions, (2) sequence Q9 KWR4 from Bacillus pumilus (Swiss-Prot) with 98% identity and/or deviations in nine amino acid positions, (3) sequence Q6SIX5 from Bacillus pumilus (Swiss-Prot) with 97% identity and/or deviations in 10 amino acid positions, (4) sequence Q5XPN0 from Bacillus pumilus (Swiss-Prot) with 97% identity and/or deviations in 11 amino acid positions.

At the level of amino acids for the mature protein, the following have been identified as the most similar: (1) sequence Q2HXI3 from Bacillus pumilus (Swiss-Prot) (SEQ ID NO:7) with 98% identity and/or deviations in five amino acid positions, (2) sequence Q6SIX5 from Bacillus pumilus (Swiss-Prot) (SEQ ID NO:5) with 98% identity and/or deviations in five amino acid positions, (3) sequence Q9 KWR4 from Bacillus pumilus (Swiss-Prot) (SEQ ID NO:6) with 98% identity and/or deviations in six amino acid positions, (4) sequence Q5XPN0 from Bacillus pumilus (Swiss-Prot) (SEQ ID NO:4) with 97% identity and/or deviations in seven amino acid positions.

On the basis of the discernible correspondences and the relationship to the other subtilisins indicated, this alkaline protease is to be regarded as a subtilisin.

One subject matter of the present invention is thus any polypeptide, in particular any hydrolase, especially any alkaline protease of the subtilisin type having an amino acid sequence which is at least 98.5% identical with the amino acid sequence given in SEQ ID NO. 2 and/or deviating in at most six amino acid positions from the amino acid sequence given in SEQ ID NO. 2.

Of these, such polypeptides whose amino acid sequence is at least 990% identical, in particular at least 99.5% identical to the amino acid sequence given SEQ ID NO. 2 are increasingly preferred, and/or those which deviate at most in five or four amino acid positions, in particular at most in three or two amino acid positions, especially preferably at most in one amino acid position with respect to the amino acid sequence given in SEQ ID NO. 2. A protein having an amino acid sequence according to SEQ ID NO. 2 is most especially preferred.

It is to be expected that their properties are very similar to those of the inventive alkali protease from B. pumilus.

As already mentioned, on the basis of a comparison of the N-terminal sequences, amino acids 1 through 108 are presumably to be regarded as the leader peptide, such that amino acids 1 through 51 presumably constitute the signal peptide, and the mature protein presumably extends from positions 109 through 383 according to SEQ ID NO. 2. Position 384 is thus taken by a stop codon, so it actually does not correspond to any amino acid. However, information about the end of a coding region may also be regarded as an important component of an amino acid sequence, so this position is included according to the present invention in the region which corresponds to the mature protein.

Another subject matter of the present invention is thus any polypeptide, in particular any hydrolase, especially any alkaline protease of the subtilisin type having an amino acid sequence which is at least 98.5% identical to the amino acid sequence given in SEQ ID NO. 2 from position 109 through position 383 (SEQ ID NO:10) and/or deviating in at most four amino acid positions from this amino acid sequence.

Of these, especially preferred polypeptides are those in which the amino acid sequence is at least 99%, especially preferably at least 99.5% identical to the amino acid sequence given in SEQ ID NO. 2 from position 109 to position 383 and/or those in which the amino acid sequence deviates in at most three amino acid positions, in particular at most two amino acid positions, especially preferably at most one amino acid position with respect to the amino acid sequence given in SEQ ID NO. 2. Most especially preferred is a protein with an amino acid sequence from position 109 to position 383 according to SEQ ID NO. 2.

If it is found, e.g., by N-terminal sequencing of the proteolytic protein released in vivo by Bacillus pumilus, that the splice site is not between amino acids 108 and 109 according to SEQ ID NO. 2 but instead is in a different location, then these statements refer to the actual splice site and/or the actual mature protein.

An additional subject matter of the present invention also includes fragments of the mature protein, in particular if they are novel in comparison with the prior art.

An additional subject matter of the present invention is therefore also polypeptides comprising an amino acid sequence with at least 100 successive amino acids, preferably at least 110, 120, 130 or 140 successive amino acids of the amino acid sequence, especially preferably at least 150, 175 or 200, most especially at least 225 or 250 successive amino acids of the amino acid sequence, from position 109 to 383 according to SEQ ID NO. 2.

An additional subject matter of the present invention is therefore also polypeptides having an amino acid sequence with at least 185 successive amino acids from position 109 to 383 according to SEQ ID NO. 2, preferably at least 190, 200 or 210, most especially at least 220, 230 or 250, or deviating therefrom in at most one amino acid position.

An additional subject matter of the present invention is therefore also polypeptides having an amino acid sequence with at least 240 successive amino acids from position 109 to 383 according to SEQ ID NO. 2, preferably at least 245, 250 or 255, most especially at least 260, 265 or 270, or deviating therefrom in at most two amino acid positions, preferably at most one amino acid position.

An additional subject matter of the present invention therefore also includes polypeptides comprising an amino acid sequence with at least 245 successive amino acids from positions 109 to 383 of the sequence given in SEQ ID NO. 2, preferably at least 250 or 255, especially preferably at least 260 or 270 successive amino acids, or deviating therefrom in at most three positions, preferably at most two positions, especially preferably at most one position.

Another subject matter of the present invention therefore also includes polypeptides comprising an amino acid sequence from position 207 to position 378 of the sequence given in SEQ ID NO. 2 (SEQ ID NO:11) or differing therefrom in at most four positions, preferably at most three, especially preferably at most two positions, especially at most in one position.

Since the signal peptide and the propeptide also have units which are of inventive interest as such, another subject matter of the present invention includes such peptides which are homologous with these polypeptides inasmuch as they are novel. As stated, amino acids 1 to 108 are presumably the leader peptide, amino acids 1 to 51 are presumably the signal peptide and accordingly, amino acids 52 to 108 are the propeptide. Another subject matter of the present invention therefore comprises polypeptides having an amino acid sequence from position 1 to position 51 (SEQ ID NO:8) as well as from position 1 to position 108 (SEQ ID NO:9) according to SEQ ID NO. 2 as well as polypeptides deviating from these amino acid sequences in one amino acid position.

Another subject matter of the present invention comprises polypeptides that are coded for by the inventive polynucleotides defined below.

This more preferably includes such polypeptides which are derived from the nucleotide sequence that is as similar as possible to the nucleotide sequence given in SEQ ID NO. 1, in particular over the partial area corresponding to positions 109 to 384 of the polypeptide according to SEQ ID NO. 2.

It is to be expected that these nucleic acids will code for proteins whose properties are increasingly similar to those of the inventive alkaline protease from B. pumilus, in particular the mature protein. Here again, as is the case for all the following embodiments, it is true that these statements refer to the actual mature protein, if it should be found that the splice site of the protein is situated at a location other than that indicated above.

The most preferred embodiment of this inventive subject matter is thus any alkaline protease of the subtilisin type, in which the amino acid sequence is identical on the whole to the amino acid sequence given in SEQ ID NO. 2, preferably in positions 109 to 383, and/or in which the amino acid sequence can be derived from the nucleotide sequence in SEQ ID NO. 1, preferably from positions 325 to 1152.

This is the case with the newly discovered alkaline protease from the Bacillus pumilus made available with the present patent application.

This is a protease not yet known in the prior art. As indicated in the examples, it is isolatable, producible and usable. As also documented in the examples, it is additionally characterized in that it at least approaches and/or even exceeds the performance of the established enzymes used for this purpose when used in a suitable medium.

The inventive polypeptides are preferably enzymes, especially preferably hydrolases, in particular proteases, especially preferably endopeptidases, in particular proteases of the subtilisin type or parts thereof. The inventive polypeptides are therefore preferably capable of hydrolyzing acid amide linkages of proteins, in particular those in the interior of proteins. The parts of the polypeptides may in particular be protein domains that may be suitable, e.g., for forming functional chimeric enzymes.

For development of industrial proteases that can be used in detergents in particular, as a naturally (microbially) formed enzyme, it may serve as a starting point, to be optimized for the desired application by essentially known mutagenesis methods, e.g., point mutagenesis, fragmentation, deletion, insertion or fusion with other proteins or protein parts of other modifications. Such optimizations may include, for example, adaptation to temperature influence, pH fluctuations, redox ratios and/or other influences, which are relevant for the industrial fields of use. For example, an improvement in oxidation stability, stability with respect to denaturing agents or proteolytic degradation, with respect to high temperatures, acidic or strongly alkaline conditions, a change in sensitivity to calcium ions or other cofactors, a reduction in immunogenicity or the allergenic effect may be desired.

Through targeted point mutations, for example, the surface charges or the loops involved in the catalysis or substrate linkages may be altered for this purpose. A starting point for this is an alignment with known proteases. This makes it possible to discover positions through whose change an improvement in the properties of the protein might be achieved, if necessary.

The mutagenesis methods are based on the respective nucleotide sequence, which is given in SEQ ID NO. 1 and/or the nucleotide sequences which are sufficiently similar thereto and are explained further below as a separate subject matter of the present invention. Corresponding methods of molecular biology are described in the prior art, e.g., in handbooks such as the one by Fritsch, Sambrook and Maniatis “Molecular Cloning: A Laboratory Manual,” Cold Spring Harbour Laboratory Press, New York, 1989.

Therefore, in addition to the protein variants based on point mutation and/or substitution mutation already mentioned above as being inventive, other embodiments of the present invention therefore also include all the aforementioned inventive polypeptides, in particular polypeptides with an amino acid sequence according to SEQ ID NO. 2 and/or from position 109 to position 383 according to SEQ ID NO. 2, polypeptides derived by insertion mutagenesis and/or substitution mutagenesis and/or inversion mutagenesis and/or by fusion with at least one other protein or protein fragment, in particular such polypeptides with insertions and/or deletions and/or inversions of up to 50 amino acids, especially preferably up to 40, 30 or 20 amino acids, in particular up to 15, 10 or five, especially up to four, three or two amino acids, especially with deletions and/or insertions of exactly one amino acid.

For example, it is thus possible to delete individual amino acids at the termini or in the loops of the enzyme without thereby loosing the proteolytic activity. Such mutations are described in WO 99/49057, for example. WO 01/07575 teaches that through such deletions, the allergenicity of the respective proteases can be decreased and thus their usability improved on the whole. The fragmentation benefits the aspect of the insertion mutagenesis or substitution mutagenesis and/or fusion with other enzymes to be described below. With regard to the intended use of these enzymes, it is especially preferred if they have a proteolytic activity even after fragmentation or deletion mutagenesis.

Numerous prior art documents also disclose advantageous effects of insertions and substitutions in subtilases. In principle, in addition to substitution of individual amino acids, substitution of multiple cohesive amino acids together also belongs here. Novel combinations of larger enzyme sections, such as the aforementioned fragments, with other proteases or proteins of a different function, also belong here. Thus, for example, based on WO 99/57254, it is possible to provide an inventive protein or parts thereof with binding domains from other proteins, e.g., cellulose binding domains via peptidic linkers or directly as fusion protein and to thereby make hydrolysis of the substrate more effective. Likewise, inventive proteins may also be linked with amylases or cellulases, for example, to exert a double function.

Of the inventive polypeptides, protein variants having one or more amino acid exchanges in positions 3, 4, 36, 42, 47, 56, 61, 69, 87, 96, 99, 101, 102, 104, 114, 118, 120, 130, 139, 141, 142, 154, 157, 188, 193, 199, 205, 211, 224, 229, 236, 237, 242, 243, 255 and 268 in the enumeration of alkaline protease from Bacillus lentus are also preferred.

Inventive chimeric proteins have a proteolytic activity in the broadest sense. This may be exerted or modified by a part of a molecule derived from an inventive polypeptide. The chimeric proteins may also be outside of the range claimed above beyond their total length. The purpose of such a fusion consists, for example, of inserting or modifying a certain function or subfunction with the help of the inventive protein part to be fused thereto. It is irrelevant in the sense of the present invention whether such a chimeric protein consists of a single polypeptide chain or multiple subunits. To implement the latter alternative, it is possible, for example, to break down a single chimeric polypeptide chain into multiple chains by a targeted proteolytic cleavage post-translationally or only after a purification step.

For example, on the basis of WO 99/57254, it is possible to provide an inventive polypeptide or parts thereof with binding domains from other proteins, e.g., the cellulose binding domains via peptidic linkers or directly as a fusion protein and thereby make hydrolysis of the substrate more effective. Such a binding domain might originate from a protease, e.g., to strengthen the binding of the inventive protein to a protease substrate. This increases the local protease concentration, which may be advantageous in individual applications, e.g., in the treatment of raw materials. Likewise, inventive proteins may also be linked to amylases or cellulases, for example, to exert a double function.

The inventive polypeptides obtainable by insertion mutation are to be classified as the inventive chimeric proteins because of their fundamental similarity. This also includes substitution variants, i.e., those in which individual regions of the molecule have been replaced by elements from other proteins.

As in formation of a hybrid, the purpose of insertion mutagenesis and substitution mutagenesis is to combine individual properties, functions or subfunctions of inventive proteins with those of other proteins. This also includes variants to be obtained, for example, by shuffling or recombination of subsequences from different proteases. In this way, proteins which have not previously been described can be obtained. Such techniques allow drastic effects or even very subtle activity modulations.

Such mutations are preferably performed according to a random method, which is to be classified as directed evolution, e.g., according to the StEP method (Zhao et al. (1998), Nat. Biotechnol., vol. 16, pp. 258-261), random priming recombination (Shao et al. (1998), Nucleic Acids Res., vol. 26, pp. 681-683), DNA shuffling (W. P. C. Stemmer (1994), Nature, vol. 370, pp. 389-391) or recursive sequence recombination (RSR; WO 98/27230, WO 97/20078, WO 95/2262) or the RACHITT method (W. M. Coco et al. (2001), Nat. Biotechnol., vol. 19, pp. 354-359). Such methods are expediently linked to a selection method or screening method which follows mutagenesis and expression to recognize variants having the desired properties. Since these techniques are performed on the DNA level, the starting point for biotechnological production is available with the respective newly created genes.

Inversion mutagenesis, i.e., a partial sequence reversal, can be regarded as a special form of deletion and also of insertion. Such variants may be created randomly or in a targeted manner.

All such inventive polypeptides that have been explained so far and are characterized in that they are capable of hydrolyzing proteins are preferred.

Such polypeptides are combined under 3.4 (peptidases) according to the official Enzyme Nomenclature 1992 of IUBMB. Of these, endopeptidases, especially groups 3.4.21 serine proteinases, 3.4.22 cysteine proteinases, 3.4.23 aspartate proteinases and 3.4.24 metalloproteinases are preferred. Of these, serine proteinases (3.4.21) are especially preferred, including subtilases and, of them, most especially subtilisins (cf. “Subtilases: subtilisin-like proteases” by R. Siezen, pages 75-95 in “Subtilisin Enzymes,” edited by R. Bott and C. Betzel, New York, 1996). Of these, in turn the subtilisins of group IS-2, the highly alkaline subtilisins are preferred.

Active molecules are preferred over inactive molecules because in the areas of use mentioned below, the proteolysis performed is important in particular.

The fragments mentioned above also have a proteolytic activity in the broadest sense, e.g., for complexing a substrate or forming a structural element required for hydrolysis. They are preferred when they can be used for hydrolysis of another protein, when considered separately for themselves, without additional protease components having to be present. This relates to the activity that can be exerted by a protease per se; the presence of buffer substances, cofactors, etc. that may be required at the same time is not affected by this.

There is naturally an interaction of different parts of the molecule for hydrolysis of proteins in deletion mutants more than in fragments, and this occurs in fusion proteins in particular, most especially those derived from a shuffling of related proteins. If a proteolytic function in the broadest sense is thereby maintained, modified, specified or achieved for the first time, the deletion variants as well as the fusion proteins are inventive proteins. Preferred representatives of this subject matter of the invention include those capable by themselves of hydrolyzing a protein substrate without requiring the presence of additional protease components.

A preferred embodiment constitutes all such inventive polypeptides discussed so far which are characterized in that they are additionally stabilized.

Their stability is thereby increased in storage and/or during their use, e.g., in the washing process, so that their activity lasts longer and is thus enhanced. The stability of inventive proteases can be increased by coupling to polymers, for example. It requires that the proteins be bound by a chemical coupling step to such polymers before their use in corresponding media.

Stabilizations that are possible via point mutagenesis of the molecule itself are preferred because they do not require any additional work steps following protein extraction. Some suitable point mutations for this are known per se from the prior art. For example, proteases can be stabilized by exchanging certain tyrosine radicals for others.

Other possibilities include, for example:

exchange of certain amino acid residues for proline;

introduction of polar or charged groups on the surface of the molecule;

change in binding of metal ions, in particular the calcium binding sites.

According to the patent U.S. Pat. No. 5,453,372, proteins can be protected from the influence of denaturing agents such as surfactants by certain mutations on the surface.

Another possibility of stabilization with respect to elevated temperature and the action of surfactants would be stabilization via exchange of amino acids situated close to the N-terminus with those that come in contact with the remainder of the molecule via noncovalent interactions and thus make a contribution toward maintaining the globular structure.

A preferred embodiment comprises all such inventive polypeptides discussed so far that are characterized in that they are additionally derivatized.

Derivatives are understood to be such proteins that are derived from the proteins mentioned via an additional modification. Such modifications may influence, for example, the stability, substrate specificity or the binding strength to the substrate or the enzymatic activity. They may also serve to reduce the allergenicity and/or immunogenicity of the protein and thus increase its tolerability, for example.

Such derivatizations may be accomplished biologically, for example, e.g., in conjunction with protein biosynthesis by the producing host organism. Coupling of low-molecular compounds such as lipids or oligosaccharides are to be emphasized in particular here.

However, derivatizations may also be performed chemically, e.g., by chemical conversion of a side chain or by covalent binding of another compound, e.g., a macromolecular compound to the protein. For example, coupling of amines to carboxyl groups of the enzyme to alter the isoelectric point may take place in this way. Furthermore, macromolecules such as proteins may be bound to inventive proteins via bifunctional chemical compounds, for example. Such a macromolecule may be, for example, a binding domain. Such derivatives are suitable in particular for use in detergents or cleaning agents. Similarly, protease inhibitors may also be bound to the inventive proteins via linkers, in particular amino acid linkers. Couplings to other macromolecular compounds, e.g., polyethylene glycol, improve the molecule with regard to additional properties such as stability or skin tolerability.

Derivatives of inventive proteins may also be understood in the broadest sense to be preparations of these enzymes. A protein may be associated with various other substances, e.g., from the culture of the producing microorganisms, depending on the production, workup or preparation. A protein may also have been mixed with certain other substances in a targeted manner, e.g., to increase its stability and storage. Therefore, all preparations of an inventive protein are also inventive. This is also independent of whether or not it actually manifests this enzymatic activity in a certain preparation because it may be desirable for it to have little or no activity in storage and to manifest its proteolytic function only at the point in the time of use. This can be controlled, for example, through suitable accompanying substances such as protease inhibitors.

A preferred embodiment includes all proteins, protein fragments, fusion proteins or derivatives that are characterized in that they have at least one antigenic determinant with one of the inventive polypeptides described above.

The secondary structural elements of a protein and its three-dimensional folding are decisive for the enzymatic activity. Domains that deviate definitely from one another in their primary structure may form largely corresponding structures spatially and may thus enable the same enzymatic behavior. Such commonalities in the secondary structure are usually recognized as corresponding antigenic determinants of antisera or pure or monoclonal antibodies. Proteins or derivatives similar to one another may thus be detected and assigned on the basis of immunochemical cross-reactions. The protective scope of the present invention therefore includes precisely such proteins, which can be assigned to the inventive proteins, protein fragments, fusion proteins or derivatives defined above, not via their homology values in the primary structure but via their immunochemical relationship to those defined above.

A preferred embodiment includes all such inventive polypeptides mentioned so far, which are characterized in that they are obtainable from a natural sources, in particular from a microorganism.

These may be unicellular fungi or bacteria, for example, because they are usually easier to produce and handle than multicellular organisms or the cell cultures derived from multicellular organisms, although the latter may constitute appropriate options for specific embodiments and thus are not fundamentally excluded from the subject matter of the invention.

Although it is possible that naturally occurring producer organisms may produce an inventive enzyme but only express it and/or secrete it into the ambient medium to a minor extent under the conditions initially ascertained, this does not rule out that suitable ambient conditions or other factors may be ascertained experimentally such that under their influence they can be stimulated to economically appropriate production of the inventive protein. Such a regulatory mechanism may be used in a targeted manner for biotechnological production. Should this also be impossible, they may still serve to isolate the respective gene.

Of these, those of gram-positive bacteria are especially preferred.

That is because they do not have any external membrane and thus the proteins secreted are released directly into the ambient medium.

Most especially preferred are those of gram-positive bacteria of the Bacillus genus.

Bacillus proteases have favorable properties for various possible technical applications from the beginning. These include a certain stability with respect to elevated temperature, oxidizing or denaturing agents. Furthermore, there is the greatest experience with microbial proteases with regard to their biotechnological production as pertaining to, for example, construction of favorable cloning vectors, selection of host cells and growth conditions or estimating risks such as the allergenicity. Bacilli are also established as producer organisms with an especially high production output in industrial processes. The wealth of experience gained in production and use of these proteases also benefits further developments of these enzymes according to the present invention. For example, this pertains to their compatibility with other chemical compounds, e.g., the ingredients of detergents or cleaning agents.

Of those from the Bacillus species, those from the Bacillus pumilus species, in particular from the strain of Bacillus pumilus used according to the present invention, are again preferred.

The embodiment of the inventive enzyme was originally obtained from these species. The respective sequences thereof are given in the sequence protocol. The variants described above can be produced from this strain or from related strains in particular by using the standard methods of molecular biology such as PCR and/or essentially known mutagenesis methods.

Another solution to the problem and thus a separate subject matter of the invention are the nucleic acids which serve to implement the invention.

By using methods that are generally known today such as chemical synthesis or the polymerase chain reaction (PCR) in combination with the standard methods of molecular biology and/or protein chemistry, it is possible for those skilled in the art to produce complete genes on the basis of known DNA sequences and/or amino acid sequences. Such methods are known from “Lexikon der Biochemie” [Lexicon of Biochemistry], Spektrum Akademischer Verlag, Berlin, 1999, vol. 1, pp. 267-271 and vol. 2, pp. 227-229, for example. This is possible in particular when a strain deposited with a strain collection can be accessed. For example, with PCR primers which are synthesizable on the basis of a known sequence and/or by means of isolated mRNA molecules, the respective genes can be synthesized, cloned and processed further, if desired, e.g., mutagenized, from such strains.

Nucleic acids form the starting point of almost all research and further developments in molecular biology as well as the production of proteins. These include in particular sequencing of genes and deriving the respective amino acid sequence, any type of mutagenesis (see above) and expression of proteins.

Mutagenesis for development of proteins having certain properties is also referred to as “protein engineering.” Properties for which they are optimized have already been given above as examples. Such a mutagenesis may be performed in a targeted manner or by random methods, e.g., using on the cloned genes a subsequent recognition method and/or selection method (screening and selection) directed at the activity, e.g., by hybridization with nucleic acid probes, or on the gene products, the proteins, e.g., via their activity. Further development of the inventive proteases may also be directed at the considerations presented in the publication “Protein engineering” by P. N. Bryan (2000) in Biochim. Biophys. Acta, vol. 1543, pp. 203-222.

Another subject matter of the present invention therefore also includes polynucleotides that code for inventive polypeptides, in particular hydrolases, especially alkaline proteases of the subtilisin type. The subject matter of the present invention therefore also includes in particular polynucleotides selected from the group comprising:
a) polynucleotide having a nucleic acid sequence according to SEQ ID NO. 1,
b) polynucleotide having a nucleic acid sequence from positions 1 to 153 according to SEQ ID NO. 1 (SEQ ID NO:12),
c) polynucleotide having a nucleic acid sequence from positions 1 to 324 according to SEQ ID NO. 1 (SEQ ID NO:13),
d) polynucleotide having a nucleic acid sequence from positions 325 to 1152 according to SEQ ID NO. 1 (SEQ ID NO:14),
e) polynucleotide coding for a polypeptide having an amino acid sequence according to SEQ ID NO. 2,
f) polynucleotide coding for a polypeptide having an amino acid sequence from positions 1 to 51 according to SEQ ID NO. 2 (SEQ ID NO:8),
g) polynucleotide coding for a polypeptide having an amino acid sequence from positions 1 to 108 according to SEQ ID NO. 2 (SEQ ID NO:9),
h) polynucleotide coding for a polypeptide having an amino acid sequence from positions 109 to 383 according to SEQ ID NO. 2 (SEQ ID NO:10),
i) polynucleotide coding for an inventive polypeptide,
j) naturally occurring or artificially created mutants or polymorphic forms or alleles of a polynucleotide according to (a) having up to 55 mutations, preferably up to 50, 45, 40 or 30, especially preferably up to 25, 20, 15 or 10, in particular up to 9, 8, 7, 6, 5, 4, 3 or 2 mutations, especially with exactly one mutation,
k) naturally occurring or artificially created mutants or polymorphic forms or alleles of a polynucleotide according to (b) or (c) having up to 8 mutations, preferably up to 7, 6 or 5, especially preferably up to 4, 3 or 2 mutations, especially with exactly one mutation,
l) naturally occurring or artificially created mutants or polymorphic forms or alleles of a polynucleotide according to (d) having up to 40 mutations, preferably up to 35, 30 or 25, especially preferably up to 20, 15 or 10, in particular up to 9, 8, 7, 6, 5, 4, 3 or 2 mutations, especially with exactly one mutation,
m) polynucleotides having a sequence homology or identity of at least 95%, preferably at least 96% or 97%, especially preferably at least 98%, especially at least 99% with respect to a polynucleotide according to (a),
n) polynucleotides having a sequence homology or identity of at least 95% with respect to a polynucleotide according to (b),
o) polynucleotides having a sequence homology or identity of at least 98% with respect to a polynucleotide according to (c),
p) polynucleotides having a sequence homology or identity of at least 95.5%, preferably at least 96 or 97%, especially preferably at least 98%, especially at least 99% with respect to a polynucleotide according to (d),
q) polynucleotides hybridizing with a polynucleotide according to (a), (b), (c) or (d) under stringent conditions, whereby the term “stringent conditions” is preferably to be understood as incubation at 60° C. in a solution containing 0.1×SSC and 0.1% sodium dodecyl sulfate (SDS), whereby 20×SSC denotes a solution containing 3M sodium chloride and 0.3M sodium citrate (pH 7.0),
r) polynucleotides comprising at least 200, in particular at least 250, 300, 350 or 400 successive nucleic acids, especially preferably at least 450, 500, 550 or 600, especially at least 650, 700, 750 or 800 successive nucleic acids of a polynucleotide according to (a), (c), (d), (g), (h), (m) or (p),
s) polynucleotides having deletions and/or insertions and/or inversions of up to 50 nucleotides, preferably up to 40, 30 or 20, especially preferably up to 15, 10 or 5, in particular up to 4, 3 or 2 nucleotides, especially insertions and/or deletions of exactly one nucleotide with respect to a polynucleotide according to (a) through (r), in particular with respect to a polynucleotide according to (a) or (d),
t) polynucleotides comprising at least one of the polynucleotides mentioned under (a) through (s),
u) polynucleotides that are complementary with polynucleotides according to (a) through (t).

The polynucleotides may be in the form of a single strand or a double strand. The subject matter of the invention also includes, in addition to the deoxyribonucleic acids, the homologous and complementary ribonucleic acids.

The subject matter of the present invention also includes in particular those polynucleotides in which certain regions have been replaced by other regions to enable expression of the inventive polypeptide, taking into account the different codon usage of a host organism used for expression.

According to the statements made above, of the inventive nucleic acids described above, the following are increasingly preferred:
those that are characterized in that they are obtainable from a natural source, in particular from a microorganism;
including those that are characterized in that the microorganism is a gram-positive bacterium;
including those that are characterized in that the gram-positive bacterium is one of the Bacillus genus, and
including those that are characterized in that the Bacillus species is Bacillus pumilus, in particular the strain used according to the present invention.

Vectors containing one of the aforementioned inventive nucleic acid regions, in particular one that codes for one of the inventive polypeptides mentioned above constitute a separate subject matter of the invention.

To allow handling of the nucleic acids relevant to the invention and thus in particular to prepare for production of inventive polypeptides, they are suitably ligated into vectors. Such vectors as well as the respective working methods are described in detail in the prior art. Vectors are obtainable commercially in large numbers and in a wide range of variation, for both cloning and expression. These include, for example, vectors derived from bacterial plasmids, bacteriophages or from viruses or predominantly synthetic vectors. Furthermore, they are differentiated according to the type of cell types in which they are capable of being established, e.g., according to vectors for gram-negative bacteria, for gram-positive bacteria, for yeasts or for higher eukaryotic organisms. They form suitable starting points, e.g., for research in molecular biology and biochemistry and for expression of the respective gene or the respective protein.

In one embodiment, the inventive vectors are cloning vectors.

Cloning vectors are suitable not only for storage, biological amplification or secretion of the gene of interest for its characterization according to molecular biology. They are at the same time forms of the claimed nucleic acids that can be shipped and stored well and also constitute the starting points for the methods of molecular biology; they are not bound to cells such as PCR or in vitro mutagenesis methods, for example.

In one embodiment, the inventive vectors are preferably expression vectors.

Such expression vectors are the basis for implementing the corresponding nucleic acids in biological production systems and thus producing the respective proteins. Preferred embodiments of this subject matter of the invention include expression vectors which carry the genetic elements required for expression, e.g., the natural promoter originally localized upstream from this gene or a promoter from another organism. These elements may be arranged in the form of a so-called expression cassette, for example. Alternatively, individual regulation elements or all regulation elements may also be made available by the respective host cell. The expression vectors are especially preferably adjusted to additional properties such as the optimal number of copies, the selected expression system, in particular the host cell (see below).

It is also advantageous for a high expression rate if the expression vector preferably contains only the respective gene as an insert and does not have any large 5′ or 3′ noncoding regions. Such inserts are obtained, for example, if the fragment obtained after random treatment of the chromosomal DNA of the starting strain with a restriction enzyme has been spliced again in a targeted manner after sequencing and before integration into the expression vector.

One example of an expression vector is the vector pAWA22. Other vectors are available to those skilled in the art from the prior art and are offered commercially in large numbers.

Cells containing an inventive polynucleotide after being modified by the methods of genetic engineering form a separate subject matter of the invention.

These cells contain the genetic information for synthesis of an inventive protein. In contrast with the natural producer organisms described above and also claimed, this includes cells which have been provided with the inventive nucleic acids according to essentially known methods and/or which are derived from such cells. Such host cells that are comparatively easy to culture and/or give high product yields are suitably selected for this purpose.

They allow, for example, amplification of the corresponding genes but also their mutagenesis or transcription and translation and ultimately biotechnological production of the respective proteins. This genetic information may be present either extra chromosomally as a separate genetic element, i.e., in bacteria in plasmidal localization, or may be integrated into a chromosome. The choice of a suitable system will depend on the questions to be answered such as the type and duration of storage of the gene and/or of the organism or the type of mutagenesis or selection. Thus mutagenesis methods and selection methods based on bacteriophages—and their specific host cells—are described in the prior art, for example, for development of detergent enzymes.

The inventive polynucleotide is preferably part of one of the inventive vectors designated above, in particular a cloning vector or an expression vector.

In this way, they become relevant for implementation of the present invention.

In addition, such cells which express and preferably secrete an inventive polypeptide are preferred.

Only the host cells that form the proteins make possible their biotechnological production. In principle all organisms, i.e., prokaryotic cells, eukaryotic cells or cyanophytes are suitable as host cells for protein expression. Preferred are such host cells that can be handled well genetically, which pertains to, for example, transformation with the expression vector, its stable establishment and regulation of expression, e.g., unicellular fungi or bacteria. Furthermore, preferred host cells are characterized by good microbiological and biotechnological handleability. For example, this pertains to easy culturability, high growth rates, low demands with regard to fermentation media and good production rates and secretion rates for foreign proteins. Laboratory strains directed at expression are preferably selected. These are available commercially or via generally accessible strain collections. Each inventive protein can be obtained theoretically in this way from a plurality of host organisms. From the abundance of different systems available according to the prior art, the optimal expression systems for the individual case must be ascertained experimentally.

Especially advantageous are host cells which are themselves protease-negative and thus do not degrade the proteins that are formed.

Preferred embodiments include those host cells whose activity is regulable on the basis of corresponding genetic elements, e.g., through controlled addition of chemical compounds, by changing the culturing conditions or as a function of the respective cell density. This controllable expression allows a very economical production of the proteins in question; it is implementable, for example, via a corresponding element on the respective vector. The gene, expression vector and host cell are suitably coordinated with one another, which pertains to the genetic elements required for expression (ribosome binding site, promoters, terminators) or codon usage.

Of these, expression hosts which secrete the protein that is formed into the ambient medium are preferred, because this allows comparatively simple processing.

Also preferred host cells which are bacteria.

Bacteria are characterized by short generation times and low demands regarding the culturing conditions. Therefore, inexpensive methods can be established. In addition, there is a great wealth of experience with bacteria in fermentation technology. For a specific production, gram-negative or gram-positive bacteria may be suitable for a wide variety of reasons to be ascertained experimentally in the individual case, such as nutrient sources, product formation rate, time required, etc.

In a preferred embodiment, it pertains to a gram-negative bacterium, in particular one of the genera Escherichia coli or Klebsiella, in particular strains of E. coli K12, E. coli B or Klebsiella planticola, and most especially derivatives of the strains Escherichia coli BL21 (DE3), E. coli RV308, E. coli DH5α, E. coli JM109, E. coli XL-1 or Klebsiella planticola (Rf).

In the case of gram-negative bacteria such as E. coli, a plurality of proteins are secreted into the periplasmic space. This may be advantageous for specific applications. In the patent application WO 01/81597, a method is disclosed for achieving the result that even gram-negative bacteria secrete the expressed proteins. Such a system is also suitable for production of the inventive proteins. The gram-negative bacteria mentioned as preferred are usually easily accessible, i.e., commercially or via public strain collections, and can be optimized for specific production conditions in conjunction with other components that are also available in large numbers such as vectors.

An alternative embodiment that is no less preferred involves a gram-positive bacterium, in particular one of the genera Bacillus, Staphylococcus or Corynebacteria, most especially of the species Bacillus lentus, B. licheniformis, B. amyloliquefaciens, B. subtilis, B. globigii, B. gibsonii, B. pumilus or B. alcalophilus, Staphylococcus carnosus or Corynebacterium glutamicum.

Gram-positive bacteria have the fundamental difference in comparison with gram-negative bacteria that they deliver the secreted proteins directly to the culture medium surrounding the cells; if desired, the expressed inventive proteins can be purified directly from the culture medium. Furthermore, they are related or identical to most of the source organisms for industrially important subtilisins and usually form comparable subtilisins themselves, so that they have a similar codon usage and their protein synthesis apparatus is naturally aligned accordingly. Another advantage may consist of the fact that by means of this method, a mixture of inventive proteins with the subtilisins formed endogenously by the host strains can be obtained. Such coexpression is also disclosed in the patent application WO 91/02792. If this is not desired, the protease genes naturally present in the host cell would have to be permanently or temporarily inactivated.

Also preferred are host cells, which are eukaryotic cells, preferably of the Saccharomyces genus.

Examples of these include fungi such as actinomycetes or even yeasts such as Saccharomyces or Kluyveromyces. Thermophilic fungal expression systems are presented in WO 96/02653 A1, for example. Such systems are suitable in particular for expression of temperature-stable variants. The modifications which implement eukaryotic systems, especially in conjunction with protein synthesis, include, for example, binding of low-molecular compounds such as membrane anchors or oligosaccharides. Such oligosaccharide modifiers may be desirable, for example, to reduce allergenicity. Coexpression with the enzymes naturally formed by such cells, e.g., cellulases, may also be advantageous.

Methods of producing an inventive polypeptide constitute an independent subject matter of the invention.

This includes the method for producing an inventive polypeptide as described above, e.g., chemical synthesis methods.

On the other hand, however, all the production methods of molecular biology, microbiology and/or biotechnology that have already been mentioned above in individual aspects and have become established in the prior art, based on the inventive nucleic acids cited above, are preferable. According to what is said above, the nucleic acids identified in the sequence protocol under SEQ ID NO. 1 or mutants or subsequences thereof derived from them accordingly may be used for this.

These are preferably methods which are performed using a previously identified vector and especially preferably using a previously identified cell, advantageously a genetically modified cell. In this way, the preferred genetic information is made available accordingly in a form that can be utilized microbiologically.

Embodiments of the present invention may also be cell-free expression systems on the basis of the respective nucleic acid sequences in which the protein biosynthesis is reproduced in vitro. All the elements already mentioned above may also be combined to yield novel methods to produce inventive proteins. A plurality of possible combinations of process steps may be conceivable for each inventive protein, so that optimal methods have to be ascertained experimentally for each individual concrete case.

According to what was said above, of the aforementioned methods, those in which the nucleotide sequence has been adapted in one codon, preferably in several codons, to the codon usage of the host strain are preferred.

A separate subject matter of the invention includes agents containing the aforementioned inventive polypeptides.

All types of agents, in particular mixtures, recipes, solutions, etc. whose usability is improved by adding one of the inventive proteins described above are included within the scope of protection of the present invention. Depending on the field of use, these may be solid mixtures, e.g., powders, with freeze-dried or encapsulated proteins or gelatinous or liquid agents. Preferred recipes contain, for example, buffer substances, stabilizers, reactants and/or cofactors of the proteases and/or other ingredients that are synergistic with the proteases. These include in particular agents for the fields of use mentioned below. Additional fields of use are derived from the prior art and are discussed in the handbook “Industrial Enzymes and Their Applications” by H. Uhlig, Wiley-Verlag, New York, 1998, for example.

Possible fields of use here include in particular use for production or treatment of raw materials or intermediates in textile production, in particular for removing dirt layers on fabrics, in particular on wool or silk, as well as use for care of textiles containing natural fibers, in particular wool or silk.

Natural fibers in particular, such as wool or silk, are characterized by a characteristic microscopic surface structure. This may lead to effects such as felting, which are unwanted in the long run, as explained on the example of wool in the article by R. Breier in Melliand Textilberichte of Apr. 1, 2000 (page 263). To avoid such effects, the natural raw materials are treated with inventive agents, which contribute toward, for example, smoothing the scaly surface structure based on protein structures and thus counteracting felting.

The subject matter of the invention accordingly also comprises methods for treatment of textile raw materials and for textile care in which inventive polypeptides are used in at least one of the process steps. Of these, the preferred processes for textile raw materials, fibers or textiles with natural constituents are in particular those with wool or silk. For example, these may be methods in which materials are prepared for processing to yield textiles, e.g., for antifelt finishing or, for example, methods which improve the cleaning of worn textiles by adding a care component.

Other possible fields of use include, for example:
use for biochemical analysis or for synthesis of low-molecular compounds or of proteins, preferably including use for end group determination as part of peptide sequence analysis;
use for preparation, purification or synthesis of natural substances of valuable biological materials;
use for treatment of natural raw materials, in particular for surface treatment, most especially in a method for treatment of leather, in particular for depilation of leather;
use for treatment of photographic film, in particular for removal of gelatin-containing layers or similar protective layers; and
use for production of foods or animal feeds, in particular for enzymatic treatment of soymilk and/or soymilk products.

Essentially the use of the aforementioned inventive polypeptides in all other technical fields for which it has been found to be suitable is included in the protective scope of the present patent application.

Another possible inventive application is use of the inventive polypeptides in cosmetic agents. These are understood to include all types of cleaning and care agents for human skin or human hair, in particular cleaning agents. The agent may also be a pharmaceutical agent, depending on the intended application.

Proteases also play a crucial role in the cell renewal process of the human skin (desquamation) (T. Egelrud et al., Acta Derm. Venerol., vol. 71 (1991), pp. 471-474). Accordingly, proteases are also used as bioactive components in skin care agents to support the degradation of desmosome structures, which are increased in dry skin. The use of subtilisin proteases with amino acid exchanges in positions R99G/A/S, S154D/E and/or L211D/E for cosmetic purposes is described in WO 97/07770 A1 for example. According to what was said above, inventive proteases may also be developed further via the corresponding point mutations. Inventive proteases, in particular those whose activity is controlled, e.g., by mutagenesis or by adding corresponding substances that interact with them, are thus also suitable as active components in skin or hair cleaning or care agents. Especially preferred are such preparations of these enzymes which, as described above, are stabilized, e.g., by coupling to macromolecular carriers (cf. U.S. Pat. No. 5,230,891) and/or that have been derivatized by point mutations at highly allergenic positions, so that they have a greater skin tolerability for humans.

Examples of inventive cosmetic and/or pharmaceutical agents include shampoos, soaps, washing lotions, creams, peeling agents, as well as oral, dental or dental prosthesis care agents. These agents may in particular also contain components such as those listed below for detergents and cleaning agents.

Accordingly, corresponding cosmetic cleaning and care methods and the use of such proteolytic enzymes for cosmetic purposes are also included in this subject matter of the invention, in particular in corresponding agents, such as shampoos, soaps or washing lotions or in care agents which are offered, e.g., in the form of creams. Use in a peeling pharmaceutical agent, e.g., for use to produce same, is also included in this subject matter.

Detergents and cleaning agents containing the inventive polypeptides are an especially preferred subject matter according to this invention because, as shown in the exemplary embodiments in the present patent application, an increase in washing performance in comparison with agents containing the proteases used traditionally has surprisingly been observed with detergents and cleaning agents using a protease that is preferred according to this invention.

The washing performance or cleaning performance of a detergent and/or cleaning agent in the sense of the present patent application is understood to refer to the effect which the agent in question has on soiled articles, e.g., textiles or objects with hard surfaces. Individual components of such agents, in particular the inventive enzymes, are evaluated with regard to their contribution to the washing performance or cleaning performance of the detergent and/or cleaning agent as a whole. To be taken into account in particular here is that it is impossible to readily deduce the contribution of an enzyme to the washing performance of an agent from its enzymatic properties. Instead, in addition to the enzymatic activity, a role is also played here in particular by factors such as stability, substrate binding, binding to the material to be cleaned or interactions with other ingredients of the detergents or cleaning agents, in particular also possible synergistic effects in the removal of soiling.

As explained above, use of the homologous proteases Q5XPN0, Q2HXI3, Q9 KWR4 and Q6SIX5 in a detergent or cleaning agent is not disclosed.

Another subject matter of the present invention therefore includes detergents and cleaning agents, in particular those containing surfactants and/or bleaching agents, which contain a polypeptide, in particular a hydrolase, preferably a protease, especially preferably an alkaline protease of the subtilisin type, selected from the group comprising:
a) polypeptide having an amino acid sequence according to SEQ ID NO. 2,
b) polypeptide having an amino acid sequence from positions 109 to 383 according to SEQ ID NO. 2 (SEQ ID NO:10),
c) naturally occurring or artificially created mutants, polymorphic forms or alleles of a polypeptide according to (a) or (b) having up to 50 mutations, more preferably having up to 45, 40, 35, 30, 25 or 20 mutations, in particular having up to 15, 12, 10, 9, 8, 7, 6 or 5 mutations, especially preferably having up to 4, 3 or 2 mutations, especially with exactly one mutation,
d) polypeptides having a sequence homology or identity of at least 80%, more preferably of at least 81%, 82%, 83%, 84%, 85%, 86%, 87%, 88%, 89% or 90%, especially preferably at least 91%, 92%, 93%, 94% or 95%, especially at least 96%. 97%, 98% or 99% with respect to a polypeptide according to (a) or (b),
e) polypeptides comprising at least 50 successive amino acids, more preferably at least 60, 70, 80, 90 or 100, especially preferably at least 120, 140, 160, 180 or 200, especially at least 220, 240, 260 or 270 successive amino acids of the amino acid sequence according to (a) or (b)
f) polypeptides having insertions and/or deletions and/or inversions of up to 50 amino acids, preferably up to 40, 30 or 20, especially preferably of up to 15, 10 or 5, in particular up to 4, 3 or 2 amino acids, especially insertions and/or deletions of exactly one amino acid with respect to a polypeptide according to (a), (b), (c), (d) or (e)
g) polypeptides comprising at least one of the polypeptides listed under (a) through (f).

The subject matter of the present invention here preferably includes detergents and cleaning agents which contain the inventive polypeptides mentioned above having a higher homology with the inventive polypeptide according to SEQ ID NO. 2 and/or with the inventive polypeptide from positions 109 to 383 according to SEQ ID NO. 2.

The inventive detergents and cleaning agents may include all conceivable types of cleaning agents, both concentrates and agents that are to be used without dilution, for use on a commercial scale, in a washing machine or in hand washing and/or hand cleaning. These include, for example, detergents for textiles, carpets or natural fibers, for which the term detergent is used according to the present invention. These also include, for example, dishwashing agents for dishwashing machines or manual dishwashing agents or cleaning agents for hard surfaces such as metal, glass, porcelain, ceramic, tiles, stone, lacquered surfaces, plastics, wood or leather; the term cleaning agent is used for such products according to the present invention. In the broader sense, sterilization agents and disinfectants may also be regarded as detergents and cleaning agents in the inventive sense.

Embodiments of the present invention include all expedient dosage forms of the inventive detergents or cleaning agents and/or those that are established according to the prior art. These include, for example, solid, powdered, liquid, gelatinous or pasty agents, optionally also comprising multiple phases, compressed or not compressed; furthermore, these also include, for example, extrudates, granules, tablets or pouches, those packaged in large drums as well as those packaged in portions.

In a preferred embodiment, the inventive detergents or cleaning agents contain the inventive polypeptides described above, in particular alkaline proteases of the subtilisin type, in an amount from 2 μg to 20 mg, preferably from 5 μg to 17.5 mg, especially preferably from 20 μg to 15 mg, most especially preferably from 50 μg to 10 mg per gram of the agent. This includes all values between these numbers, both integers and nonintegers.

The protease activity in such agents may be determined according to the method described in Tenside [Surfactants], vol. 7 (1970), pp. 125-132. The protease activity is given in PE (protease units) accordingly.

In a comparison of the performances of two detergent enzymes, as in the examples in the present patent application, for example, a distinction must be made between protein-equivalent use and activity-equivalent use. In particular in the case of preparations that are produced by genetic engineering and are largely free of secondary activity, the protein-equivalent use is indicated. This allows a statement about whether the same quantities of protein—as a measure of the yield of enzymatic production—lead to comparable results. If the respective ratios of active substance to total protein (the values of the specific activity) differ greatly, then an activity-equivalent comparison is to be recommended, because the respective enzymatic properties are compared in this way. In general, it is true that a low specific activity can be compensated by adding a larger amount of protein. This is ultimately an economic consideration.

In addition to the inventive polypeptide, an inventive detergent or cleaning agent optionally contains other ingredients such as additional enzymes, enzyme stabilizers, surfactants, e.g., nonionic, anionic and/or amphoteric surfactants and/or bleaching agents and/or builders as well as optionally other conventional ingredients which are mentioned below.

Preferably alkoxylated, advantageously ethoxylated, in particular primary alcohols, preferably with 8 to 18 carbon atoms and an average of 1 to 12 mol ethylene oxide (EO) per mol alcohol are used as the nonionic surfactants, in which the alcohol radical may be linear or preferably may have methyl branching in position 2, and/or may contain linear and methyl-branched radicals in the mixture such as those usually found in oxo alcohol radicals. In particular, however, alcohol ethoxylates having linear radicals of alcohols of native origin with 12 to 18 carbon atoms, e.g., from coco fatty alcohol, palm fatty alcohol, tallow fatty alcohol or oleyl alcohol and an average of 2 to 8 EO per mol alcohol are preferred. The preferred ethoxylated alcohols include, for example, C₁₂₋₁₄ alcohols having 3 EO or 4 EO, C₉₋₁₁ alcohols having 7 EO, C₁₃₋₁₅ alcohols having 3 EO, 5 EO, 7 EO or 8 EO, C₁₂₋₁₈ alcohols having 3 EO, 5 EO or 7 EO and mixtures thereof, such as mixtures of C₁₂₋₁₄ alcohol having 3 EO and C₁₂₋₁₈ alcohol having 5 EO. The stated degrees of ethoxylation are statistical averages which may be an integer or a fraction for a specific product. Preferred alcohol ethoxylates have a narrow homolog distribution (narrow range ethoxylates, NRE). In addition to these nonionic surfactants, fatty alcohols having more than 12 EO may also be used. Examples of these include tallow fatty alcohol having 14 EO, 25 EO, 30 EO or 40 EO.

Another class of nonionic surfactants that are preferred for use and may be used either as the exclusive nonionic surfactant or in combination with other nonionic surfactants include alkoxylated, preferably ethoxylated or ethoxylated and propoxylated fatty acid alkyl esters, preferably with 1 to 4 carbon atoms in the alkyl chain, in particular fatty acid methyl esters.

Another class of nonionic surfactants which may advantageously be used are the alkyl polyglycosides (APG). Alkyl polyglycosides that may be used conform to the general formula RO(G)_{z} in which R denotes a linear or branched, in particular methyl-branched in position 2, saturated or unsaturated aliphatic radical with 8 to 22 carbon atoms, preferably 12 to 18 carbon atoms, and G is the symbol standing for a glycose unit having 5 or 6 carbon atoms, preferably glucose. The degree of glycosylation z here is between 1.0 and 4.0, preferably between 1.0 and 2.0 and in particular between 1.1 and 1.4. Linear alkyl polyglucosides, i.e., alkyl polyglycosides in which the polyglycosyl radical is a glucose radical and the alkyl radical is an n-alkyl radical, are preferred for use here.

Nonionic surfactants of the amine oxide type, e.g., N-cocoalkyl-N,N-dimethylamine oxide and N-tallow alkyl-N,N-dihydroxyethylamine oxide and fatty acid alkanolamides may also be suitable as the nonionic surfactants. The amount of these nonionic surfactants is preferably no greater than that of the ethoxylated fatty alcohols, in particular no more than half thereof.

Other suitable surfactants include polyhydroxy fatty acid amides of formula (II)

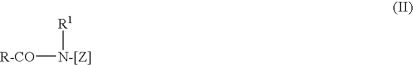

in which RCO stands for an aliphatic acyl radical with 6 to 22 carbon atoms, R¹ stands for hydrogen, an alkyl or hydroxyalkyl radical with 1 to 4 carbon atoms and [Z] stands for a linear or branched polyhydroxyalkyl radical with 3 to 10 carbon atoms and 3 to 10 hydroxyl groups. The polyhydroxy fatty acid amides are known substances which may usually be obtained by reductive amination of a reducing sugar with ammonium, an alkylamine or an alkanolamine and subsequent acylation with a fatty acid, a fatty acid alkyl ester or a fatty acid chloride.

The group of polyhydroxy fatty acid amides also includes compounds of formula (III)

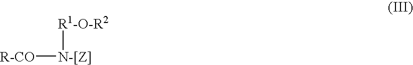

in which R stands for a linear or branches alkyl or alkenyl radical with 7 to 12 carbon atoms, R¹ stands for a linear, branched or cyclic alkyl radical or an aryl radical with 2 to 8 carbon atoms and R² stands for a linear, branched or cyclic alkyl radical or an aryl radical or an oxyalkyl radical with 1 to 8 carbon atoms, wherein C₁₋₄ alkyl or phenyl radicals are preferred and [Z] stands for a linear polyhydroxyalkyl radical whose alkyl chain is substituted with at least two hydroxyl groups, or alkoxylated, preferably ethoxylated or propoxylated derivatives of this radical.

[Z] is preferably obtained by reductive amination of a reducing sugar, e.g., glucose, fructose, maltose, lactose, galactose, mannose or xylose. The N-alkoxy- or N-aryloxy-substituted compounds may be converted to the desired polyhydroxy fatty acid amides by reaction with fatty acid methyl esters in the presence of an alkoxide as the catalyst.

Anionic surfactants used include, for example, those of the sulfonate and sulfate types. Surfactants of the sulfonate type that may be considered preferably include C₉₋₁₃ alkylbenzenesulfonates, olefinsulfonates, i.e., mixtures of alkenesulfonates and alkenedisulfonates and hydroxyalkanesulfonates and -disulfonates, such as those obtained, for example, from C₁₂₋₁₈ monoolefins having terminal or internal double bonds by sulfonation with gaseous sulfur trioxide and subsequent alkaline or acid hydrolysis of the sulfonation products. Also suitable are alkanesulfonates obtained from C₁₂₋₁₈ alkanes, e.g., by sulfochlorination or sulfoxidation with subsequent hydrolysis and/or neutralization. Likewise, the esters of α-sulfofatty acids (ester sulfonates), e.g., the α-sulfonated methyl esters of hydrogenated coco fatty acids, palm kernel fatty acids or tallow fatty acids are also suitable.

Other suitable anionic surfactants include sulfated fatty acid glycerol esters. Fatty acid glycerol esters are understood to include the monoesters, diesters and triesters as well as mixtures thereof, such as those obtained in production by esterification of a monoglycerol with 1 to 3 mol fatty acid or in transesterification or triglycerides with 0.3 to 2 mol glycerol. Preferred sulfated fatty acid glycerol esters are the sulfation products of saturated fatty acids with 6 to 22 carbon atoms, e.g., caproic acid, caprylic acid, capric acid, myristic acid, lauric acid, palmitic acid, stearic acid or behenic acid.

Preferred alk(en)yl sulfates are the alkali salts and in particular the sodium salts of sulfuric acid hemiesters of C₁₂-C₁₈ fatty alcohols, e.g., from coconut fatty alcohol, tallow fatty alcohol, lauryl alcohol, myristyl alcohol, cetyl alcohol or stearyl alcohol or C₁₀-C₂₀ oxo alcohols and the hemiesters of secondary alcohols of these chain lengths. Also preferred are alk(en)yl sulfates of the aforementioned chain length, containing a synthetic straight chain alkyl radical produced on a petrochemical basis and having a degradation behavior similar to that of the adequate compounds based on raw materials from fat chemistry. Of technical interest for detergents, the C₁₂-C₁₆ alkyl sulfates and C₁₂-C₁₅ alkyl sulfates and C₁₄-C₁₅ alkyl sulfates are preferred. Also 2,3-alkyl sulfates are suitable anionic surfactants.

The sulfuric acid monoesters of linear or branched C₇₋₂₁ alcohols ethoxylated with 1 to 6 mol ethylene oxide are also suitable, such as 2-methyl-branched C₉₋₁₁ alcohols having an average of 3.5 mol ethylene oxide (EO) or C₁₂₋₁₈ fatty alcohols having 1 to 4 EO. They are used only in relatively small amounts in cleaning agents, e.g., in amounts of up to 5 wt %, usually 1 to 5 wt %, because of their high foaming behavior.

Other suitable anionic surfactants also include the salts of alkyl sulfosuccinic acid, which are also referred to as sulfosuccinates or sulfosuccinic acid esters, and the monoesters and/or diesters of sulfosuccinic acid with alcohols, preferably fatty alcohols and in particular ethoxylated fatty alcohols. Preferred sulfosuccinates contain C₈₋₁₈ fatty alcohol radicals or mixtures thereof. Preferred sulfosuccinates contain in particular a fatty alcohol radical derived from ethoxylated fatty alcohols, which are nonionic surfactants when considered separately (see description above). Again sulfosuccinates in which the fatty alcohol radicals are derived from ethoxylated fatty alcohols having a narrow range homolog distribution are especially preferred. Likewise, it is also possible to use alk(en)yl succinic acid having preferably 8 to 18 carbon atoms in the alk(en)yl chain or the salts thereof.

As additional anionic surfactants, soaps in particular may be considered. Suitable soaps include saturated fatty acids soaps such as the salts of lauric acid, myristic acid, palmitic acid, stearic acid, hydrogenated erucaic acid and behenic acid as well as in particular soap mixtures derived from natural fatty acids, e.g., coco fatty acids, palm kernel fatty acids or tallow fatty acids.

The anionic surfactants including the soaps may be in the form of their sodium, potassium or ammonium salts and as soluble salts of organic bases such as monoethanolamine, diethanolamine or triethanolamine. The anionic surfactants are preferably in the form of their sodium or potassium salts, in particular in the form of the sodium salts.

The surfactants may be present in the inventive cleaning agents or detergents in a total amount of preferably 5 wt % to 50 wt %, in particular from 8 wt % to 30 wt %, based on the finished agent.

Inventive detergents or cleaning agents may contain bleaching agents. Of the compounds that supply H₂O₂ in water and serve as bleaching agents, sodium percarbonate, sodium perborate tetrahydrate and sodium perborate monohydrate are especially important. Other usable bleaching agents include, for example, peroxopyrophosphates, citrate perhydrates and peracid salts or peracids that supply H₂O₂ such as persulfates and/or persulfuric acid. The urea peroxohydrate percarbamide, which can be described by the formula H₂N—CO—NH₂.H₂O₂ may also be used. In particular when the agents are used for cleaning hard surfaces, e.g., in a dishwashing machine, they may, if desired, also contain bleaching agents from the group of organic bleaching agents, although their use is also possible in principle in agents for washing textiles. Typical organic bleaching agents include the diacyl peroxides, e.g., dibenzoyl peroxide. Other typical organic bleaching agents include the peroxy acids, whereby the alkyl peroxy acids and aryl peroxy acids may be mentioned as examples in particular. Preferred representatives include peroxybenzoic acid and its ring-substituted derivatives such as alkyl peroxybenzoic acids, but it is also possible to use peroxy-α-naphthoic acid and magnesium monoperphthalate, the aliphatic or substituted aliphatic peroxy acids such as peroxylauric acid, peroxystearic acid, ε-phthalimidoperoxycaproic acid (phthalimidoperoxyhexanoic acid, PAP), o-carboxybenzamidoperoxycaproic acid, N-nonenylamidoperadipic acid and N-nonenylamidopersuccinate and aliphatic and araliphatic peroxydicarboxylic acids, e.g., 1,12-diperoxycarboxylic acid, 1,9-diperoxyazelaic acid, diperoxysebacic acid, diperoxybrassylic acid, diperoxyphthalic acids, 2-decyl-diperoxybutane-1,4-dioic acid, N,N-tereph-thaloyldi-(6-aminopercaproic acid).

The bleaching agent content of the detergents or cleaning agents may amount to 1 wt % to 40 wt % and in particular 10 wt % to 20 wt %, whereby perborate monohydrate or percarbonate is advantageously used.

To achieve an improved bleaching effect when washing at temperatures of 60° C. or lower, and in particular in laundry pretreatment, the agents may also contain bleach activators. Compounds that may be used as bleach activators yield, under perhydrolysis conditions, aliphatic peroxocarboxylic acids, preferably having 1 to 10 carbon atoms, in particular 2 to 4 carbon atoms and/or optionally substituted perbenzoic acid. Suitable substances are those having O- and/or N-acyl groups of the aforementioned number of carbon atoms and/or optionally substituted benzoyl groups. Preferred are polyacylated alkylenediamines, in particular tetraacetyl-ethylenediamine (TAED), acylated triazine derivatives, in particular 1,5-diacetyl-2,4-dioxohexahydro-1,3,5-triazine (DADHT), acylated glycolurils, in particular 1,3,4,6-tetraacetylglycoluril (TAGU), N-acylimide, in particular N-nonanoylsuccinimides (NOSI), acylated phenol sulfonates, in particular n-nonanoyl- or isononanoyloxybenzenesulfonates (n- and/or iso-NOBS), acylated hydroxycarboxylic acids such as triethyl-O-acetyl citrate (TEOC), carboxylic acid anhydrides, in particular phthalic acid anhydride, isatoic acid anhydride and/or succinic acid anhydride, carboxylic acid amides such as N-methyldiacetamide, glycolide, acylated polyvalent alcohols, in particular triacetin, ethylene glycol diacetate, isopropenyl acetate, 2,5-diacetoxy-2,5-dihydrofuran and the enol esters known from German patent applications DE 196 16 693 and DE 196 16 767 as well as acetylated sorbitol and mannitol and/or their mixtures described in European Patent Application EP 0 525 239 (SORMAN), acylated sugar derivatives, in particular pentaacetylglucose (PAG), pentaacetylfructose, tetraacetylxylose and octaacetyllactose as well as acylated, optionally N-alkylated glutamine and/or gluconolactone, triazole and/or triazole derivatives and/or particulate caprolactams and/or caprolactam derivatives, preferably N-acylated lactams, e.g., N-benzoylcaprolactam and N-acetylcaprolactam, which are known from the international patent applications WO 94/27970, WO 94/28102, WO 94/28103, WO 95/00626, WO 95/14759 and WO 95/17498. The hydrophilically substituted acylacetals known from German patent application DE 196 16 770 and the acyllactams described in the International Patent Application WO 95/14075 are also preferred for use here. The combinations of conventional bleach activators known from German patent application DE 44 43 177 may also be used. Likewise, nitrile derivatives such as cyanopyridines, nitrile quats, e.g., N-alkylammonium acetonitriles and/or cyanamide derivatives may also be used. Preferred bleach activators are sodium 4-octanoyloxybenzenesulfonate, n-nonanoyl- or isononanoyloxybenzenesulfonate (n- and/or iso-NOBS), undecenoyloxybenzenesulfonate (UDOBS), sodium dodecanoyloxybenzene-sulfonate (DOBS), decanoyloxybenzoic acid (DOBA, OBC 10) and/or dodecanoyloxybenzenesulfonate (OBS 12) as well as N-methylmorpholinium acetonitrile (MMA). Such bleach activators may be present in the usual quantity range of 0.01 wt % to 20 wt %, preferably in amounts of 0.1 to 15 wt %, in particular 1 wt % to 10 wt %, based on the total composition.

In addition to or instead of the conventional bleach activators, so-called bleach catalysts may also be present. These substances are transition metal salts and/or transition metal complexes that are bleaching enhancers, such as Mn-, Fe-, Co-, Ru- or Mo-salene complexes or -carbonyl complexes. Mn, Fe, Co, Ru, Mo, Ti, V and Cu complexes with N-containing tripod ligands as well as Co-, Fe-, Cu- and Ru-amine complexes are also suitable as bleach catalysts, such compounds as those described in DE 19709284 A1 being preferably used.

Inventive detergents or cleaning agents usually contain one or more builders, in particular zeolites, silicates, carbonates, organic cobuilders and—where ecological reasons do not speak against their use—also phosphates. The latter are the preferred builders for use in cleaning agents for dishwashing machines in particular.

Crystalline sheet sodium silicates of the general formula NaMSiₓO₂ₓ₊₁.yH₂O, where M denotes sodium or hydrogen, x is a number from 1.6 to 4, preferably 1.9 to 4.0 and y is a number from 0 to 20 and preferred values for x are 2, 3 or 4 may be mentioned here. Such crystalline sheet silicates are described in European Patent Application EP 164514, for example. Preferred crystalline sheet silicates of the stated formula are those in which M stands for sodium and x assumes the values 2 or 3. In particular both β- and 5-sodium disilicates Na₂Si₂O₅.yH₂O are preferred. Commercially such compounds are available under the brand name SKS® (Clariant). SKS-6® is primarily a δ-sodium disilicate with the formula Na₂Si₂O₅.yH₂O; SKS-7® is primarily β-sodium disilicate. By reaction with acids (for example, citric acid or carbonic acid), the δ-sodium disilicate yields kanemite NaHSi₂O₅.yH₂O, which is available commercially under the brand names SKS-9® and/or SKS-10® (Clariant). It may also be advantageous to use chemical modifications of these sheet silicates. For example, the alkalinity of the sheet silicates may be influenced in a suitable manner. Sheet silicates doped with phosphate and/or with carbonate have modified crystal morphologies in comparison with δ-sodium disilicate, they dissolve more rapidly and have an increased calcium binding capacity in comparison with δ-sodium disilicate. Sheet silicates of the general empirical formula xNa₂O.ySiO₂.zP₂O₅ in which the ratio of x to y corresponds to a number from 0.35 to 0.6, the ratio of x to z corresponds to a number from 1.75 to 1200, and the ratio of y to z corresponds to a number from 4 to 2800 are described in the patent application DE 196 01 063. The solubility of the sheet silicates may also be increased by using especially finely divided sheet silicates. Compounds of the crystalline sheet silicates with other ingredients may also be used. To be mentioned here In particular are compounds with cellulose derivatives, which have advantages in the disintegrating effect and are used in detergent tablets in particular, as well as compounds with polycarboxylates, for example, citric acid, and/or polymeric polycarboxylates, for example, copolymers of acrylic acid.

Amorphous sodium silicates having a modulus of Na₂O:SiO₂ of 1:2 to 1:3.3, preferably from 1.2 to 1:2.8 and in particular from 1:2 to 1:2.6, which have delayed dissolving and secondary washing properties may also be used here. The delayed dissolving in comparison with traditional amorphous sodium silicates may be achieved in various ways, e.g., by surface treatment, compounding, compacting/compressing or by overdrying. Within the context of this invention, the term “amorphous” is also understood to be “amorphous to x-rays.” This means that in x-ray diffraction experiments, the silicates do not yield sharp x-ray reflexes such as those typical of crystalline substances, but instead yield one or more maximums of the scattered x-ray radiation having a width of several degree unit of the diffraction angle. However, it may indeed even lead to especially good builder properties if the silicate particles yield blurred or even sharp diffraction maximum in electron diffraction experiments. This is to be interpreted as that the products have microcrystalline regions of the size 10 nm up to a few hundred nm, values up to max. 50 nm and in particular up to max. 20 nm are preferred. Compressed/compacted amorphous silicates, compounded amorphous silicates and overdried x-ray amorphous silicates are preferred in particular.

A finely crystalline synthetic zeolite containing bound water that may optionally also be used is preferably zeolite A and/or zeolite P. Zeolite MAP® (commercial product of the company Crosfield) is especially preferred as zeolite P. However, zeolite X and mixtures of A, X and/or P are also suitable. Commercially available and preferred for use within the context of the present invention is also a cocrystal product of zeolite X and zeolite A, for example (approx. 80 wt % zeolite X) which is distributed by the company CONDEA Augusta S.p.A. under the brand names VEGOBOND AX® and can be described by the formula

nNaO.(1−n)K₂O.Al₂O₃.(2-2.5)SiO₂.(3.5-5.5)H₂O.

Suitable zeolites have an average particle size of less than 10 μm (volume distribution; measurement method: Coulter counter) and preferably contain 18 wt % to 22 wt % and in particular 20 wt % to 22 wt % bound water.

Use of the generally known phosphates as builder substances is of course also possible if such a use should not be avoided for ecological reasons. Of the variety of commercially available phosphates, the alkali metal phosphates have the greatest importance with special preference for pentasodium and/or pentapotassium triphosphates (sodium and/or potassium tripolyphosphate) in the detergent industry and cleaning agent industry.

Alkali metal phosphate is the general term for the alkali metal salts (in particular sodium and potassium salts) of the various phosphoric acids, of which metaphosphoric acids (HPO₃)ₙ and orthophosphoric acid H₃PO₄ may also be differentiated in addition to the higher-molecular representatives. The phosphates combine several advantages: they act as alkali carriers, prevent lime deposits on machine parts and/or lime encrustations on fabrics and also contribute toward the cleaning performance.

Sodium dihydrogen phosphate NaH₂PO₄ exists as a dihydrate (density 1.91 g·cm⁻³, melting point 60° C.) and as a monohydrate (density 2.04 g·cm⁻³). Both salts are white powders that are very readily soluble in water and loose the water of crystallization when heated, converting into the weakly acidic diphosphate (disodium hydrogen diphosphate Na₂H₂P₂O₇) at 200° C.; a higher temperature converting to sodium trimetaphosphate (Na₃P₃O₉) and Maddrell's salt (see below). NaH₂PO₄ gives an acid reaction; it is formed when phosphoric acid is adjusted to a pH of 4.5 with sodium hydroxide solution and the slurry is sprayed. Potassium dihydrogen phosphate (primary or monobasic potassium phosphate, potassium biphosphate, KDP), KH₂PO₄, is a white salt with a density of 2.33 g·cm⁻³ and a melting point of 253° C. (decomposing, forming potassium polyphosphate (KPO₃)ₓ) and is readily soluble in water.

Disodium hydrogen phosphate (secondary sodium phosphate) Na₂HPO₄ is a colorless crystalline and highly water-soluble salt. It exists in an anhydrous form and with 2 mol water (density 2.066 g·cm⁻³, water loss at 95° C.), 7 mol water (density 1.68 g cm³, melting point 48° C. with the loss of 5H₂O) and 12 mol water (density 1.52 g cm³, melting point 35° C. with the loss of 5H₂O), become anhydrous at 100° C. and is converted to the diphosphate Na₄P₂O₇ when heated more. Disodium hydrogen phosphate is produced by neutralization of phosphoric acid with sodium carbonate solution using phenolphthalein as an indicator. Dipotassium hydrogen phosphate (secondary or dibasic potassium phosphate) K₂HPO₄ is an amorphous white salt that is readily soluble in water.

Trisodium phosphate, tertiary sodium phosphate, Na₃PO₄ is colorless crystals, which, as a dodecahydrate, have a density of 1.62 g·cm⁻³ and a melting point of 73-76° C. (decomposing), as a decahydrate (corresponding to 19-20% P₂O₅) have a melting point of 100° C. and in anhydrous form (corresponding to 39-40% P₂O₅) have a density of 2.536 g·cm⁻³. Trisodium phosphate is readily soluble in water with an alkaline reaction and is prepared by evaporating a solution of exactly 1 mol disodium phosphate and 1 mol NaOH. Tripotassium phosphate (tertiary or tribasic potassium phosphate) K₃PO₄ is a white deliquescent granular powder with a density of 2.56 g·cm⁻³, has a melting point of 1340° C. and is readily soluble in water with an alkaline reaction. It is formed, e.g., by heating Thomas slag with carbon and potassium sulfate. Despite the higher price, the more readily soluble and therefore highly effective potassium phosphates are much preferred in the cleaning agent industry in comparison with the corresponding sodium compounds.

Tetrasodium diphosphate (sodium pyrophosphate) Na₄P₂O₇ exists in an anhydrous form (density 2.534 g cm³, melting point 988° C., also given as 880° C.) and as a decahydrate (density 1.815-1.836 g cm³, melting point 94° C. with loss of water). Both substances are colorless crystals that dissolve in water with an alkaline reaction. Na₄P₂O₇ is formed by heating disodium phosphate to >200° C. or by reacting phosphoric acid with sodium carbonate in a stoichiometric ratio and dehydrating the solution by spraying. The decahydrate complexes heavy metal salts and the salts that cause water hardness, and therefore reduces the hardness of water. Potassium diphosphate (potassium pyrophosphate) K₄P₂O₇ exists in the form of the trihydrate and is a colorless hygroscopic powder with the density 2.33 g·cm⁻³; it is soluble in water and the pH of the 1% solution at 25° C. is 10.4.

By condensation of NaH₂PO₄ and/or KH₂PO₄ higher-molecular sodium phosphates and potassium phosphates are formed; the cyclic representatives, the sodium metaphosphates and/or potassium metaphosphates and chain-type substances, the sodium and/or potassium polyphosphates can be differentiated. A number of terms have been used for the latter: fused or calcined phosphates, Graham's salt, Kurrol's salt and Maddrell's salt. All the higher sodium and potassium phosphates are referred to jointly as condensed phosphates.

Pentasodium triphosphate (Na₅P₃O₁₀; sodium tripolyphosphate) which is important industrially is a nonhygroscopic, white, water-soluble salt of the general formula NaO—[P(O)(ONa)—O]ₙ—Na, where n=3; it may be anhydrous or may crystallize with 6H₂O. In 100 g water at room temperature, approx. 17 g will dissolve; at 60° C. approx. 20 g; approx. 32 g of the salt that is free of water of crystallization will dissolve at 100° C.; after heating the solution for 2 hours at 100° C., approx. 8%/0 orthophosphate and 150% diphosphate are formed by hydrolysis. In production of pentasodium triphosphate, phosphoric acid is reacted with sodium carbonate solution or sodium hydroxide solution in a stoichiometric ratio and the solution is dehydrated by spraying. Like Graham's salt and sodium phosphate, pentasodium triphosphate will dissolve many insoluble metal compounds (even lime soaps, etc.). Pentapotassium triphosphate K₅P₃O₁₀ (potassium tripolyphosphate) is commercially available in the form of 50 wt % solution (>230% P₂O₅, 250% K₂O), for example. The potassium polyphosphates are widely used in the detergent and cleaning agent industry. In addition, there are also sodium potassium tripolyphosphates, which may also be used within the scope of the present invention. These are formed, for example, when sodium trimetaphosphate is hydrolyzed with KOH:

(NaPO₃)₃+2KOH→Na₃K₂P₃O₁₀+H₂O

These may be used according to the invention exactly like sodium tripolyphosphate, potassium tripolyphosphate or mixtures of these two; mixtures of sodium tripolyphosphate and sodium potassium tripolyphosphate or mixtures of potassium tripolyphosphate and sodium potassium tripolyphosphate or mixtures of sodium tripolyphosphate and potassium tripolyphosphate and sodium potassium tripolyphosphate may also be used according to the invention.

Organic cobuilders that may be used in the inventive detergents and cleaning agents include in particular polycarboxylates or polycarboxylic acids, polymeric polycarboxylates, polyaspartic acid, polyacetals, optionally oxidized dextrins, other organic cobuilders (see below) and phosphonates. These classes of substances are described below.

Organic builder substances that may be used include, for example, the polycarboxylic acids that may be used in the form of their sodium salts, whereby polycarboxylic acids are understood to be carboxylic acids having more than one acid function. For example, these include citric acid, adipic acid, succinic acid, glutaric acid, malic acid, tartaric acid, maleic acid, fumaric acid, sugar acids, aminocarboxylic acids, nitrotriacetic acid (NTA), if such a use is not to be avoided for ecological reasons, as well as mixtures of these. Preferred salts are the salts of the polycarboxylic acids such as citric acid, adipic acid, succinic acid, glutaric acid, tartaric acid, sugar acids and mixtures of these.

The acids per se may also be used. In addition to the builder effect, they typically also have the property of an acidifying component and thus also serve to adjust a lower and milder pH of the detergents or cleaning agents, if the pH resulting from mixing the other components is not desired. To be mentioned in particular here are acids which are compatible with the system and are environmentally acceptable, such as citric acid, acetic acid, tartaric acid, maleic acid, lactic acid, glycolic acid, succinic acid, glutaric acid, adipic acid, gluconic acid and any mixtures of these. However, mineral acids, in particular sulfuric acid, or bases, in particular ammonium hydroxide or alkali hydroxides, may also be used as pH regulators. Such regulators are present in the inventive agents in amounts of preferably no more than 20 wt %, in particular from 1.2 wt % to 17 wt %.

Suitable builders are also polymeric polycarboxylates, which include, for example, the alkali metal salts of polyacrylic acid or of polymethacrylic acid, e.g., those having a relative molecular weight of 500 g/mol to 70,000 g/mol.

The molecular weights given for the polymeric polycarboxylates are, in the sense of this document, weight-average molecular weights M, of the respective acid form, which have been determined fundamentally by means of gel permeation chromatography (GPC) using a UV detector. The measurement was performed against an external polyacrylic acid standard that supplies realistic molecular weight values because of its structural relationship to the polymers being investigated. The data definitely deviate from the molecular weight data using polystyrene sulfonic acids as the standard. The molecular weights measured against polystyrene sulfonic acids are usually much higher than the molecular weights given in the present document.

Suitable polymers are in particular polyacrylates, which preferably have a molecular weight of 2000 g/mol to 20,000 g/mol. From this group, the short-chain polyacrylates having molecular weights of 2000 g/mol to 10,000 g/mol and especially preferably from 3000 g/mol to 5000 g/mol may also be preferred because of their superior solubility.

Also suitable are copolymeric polycarboxylates in particular those of acrylic acid with methacrylic acid and acrylic acid or methacrylic acid with maleic acid. Copolymers of acrylic acid with maleic acid containing 50 wt % to 90 wt % acrylic acid and 50 wt % to 10 wt % maleic acid have proven to be especially suitable. Their relative molecular weight, based on free acids, is generally 2000 g/mol to 70,000 g/mol, preferably 20,000 g/mol to 50,000 g/mol and in particular 30,000 g/mol to 40,000 g/mol. The (co)polymeric polycarboxylates may be used either as a powder or as an aqueous solution. The amount of (co)polymeric polycarboxylates contained in the agents may be from 0.5 wt % to 20 wt %, in particular 1 wt % to 10 wt %.

To improve the water solubility, the polymers may also contain alkylsulfonic acids, e.g., allyloxybenzenesulfonic acid and methylallylsulfonic acid as monomers.

Biodegradable polymers of more than two different monomers units, e.g., those containing as monomers the salts of acrylic acid and maleic acid as well as vinyl alcohol and/or vinyl alcohol derivatives or containing as monomers the salts of acrylic acid and 2-alkylallylsulfonic acid as well as sugar derivatives are also preferred in particular.

Other preferred copolymers are those containing as monomers preferably acrolein and acrylic acid/acrylic acid salts and/or acrolein and vinyl acetate.

Likewise, other preferred builder substances to be mentioned include polymeric aminodicarboxylic acids, their salts or their precursor substances. Polyaspartic acids and/or their salts and derivatives are especially preferred.

Other suitable builder substances include polyacetals, which can be obtained by reacting dialdehydes with polyol carboxylic acids having 5 to 7 carbon atoms and at least three hydroxyl groups. Preferred polyacetals are obtained from dialdehydes such as glyoxal, glutaraldehyde, terephthalaldehyde and mixtures thereof and from polyol carboxylic acids such as gluconic acid and/or glucoheptonic acid.

Other suitable organic builder substances include dextrins, e.g., oligomers and/or polymers of carbohydrates which can be obtained by partial hydrolysis of starches. The hydrolysis may be performed according to conventional processes, e.g., acid-catalyzed or enzyme-catalyzed processes. These are preferably hydrolysis products having average molecular weights in the range of 400 g/mol to 500,000 g/mol. A polysaccharide having a dextrose equivalent (DE) in the range of 0.5 to 40, in particular from 2 to 30 is preferred, where DE is a conventional measure of the reducing effect of a polysaccharide in comparison with dextrose, which has a DE of 100. Both maltodextrins with a DE between 3 and 20 and dry glucose syrups with a DE between 20 and 37 as well as so-called yellow dextrins and white dextrins with higher molecular weights in the range of 2000 g/mol to 30,000 g/mol may also be preferred.

The oxidized derivatives of such dextrins are the reaction products thereof with oxidizing agents that are capable of oxidizing at least one alcohol function of the saccharide ring to the carboxylic acid function. Especially preferred organic builders for inventive agents include oxidized starches and/or their derivatives from the patent applications EP 472042, WO 97/25399 and EP 755944.

Other suitable cobuilders include oxydisuccinates and other derivatives of disuccinates, preferably ethylenediaminedisuccinate. Ethylenediamine-N,N′-disuccinate (EDDS) is preferably used in the form of its sodium or magnesium salts. Also preferred in this context are glycerol disuccinates and glycerol trisuccinates. Suitable amounts for use are between 3 wt % and 15 wt % in formulations containing zeolite, carbonate and/or silicate.

Other organic cobuilders that may also be used include, for example, acetylated hydroxycarboxylic acids and/or their salts, which may optionally also be in lactone form and which contain at least four carbon atoms and at least one hydroxyl group plus maximally two acid groups.

The phosphonates are another substance class with cobuilder properties. These include in particular hydroxyalkanephosphonates and/or aminoalkanephosphonates. Of the hydroxyalkanephosphonates, 1-hydroxy-ethane-1,1-diphosphonate (HEDP) is especially important as a cobuilder. It is preferably used as a sodium salt, in which the disodium salt gives a neutral reaction and the tetrasodium salt gives an alkaline reaction (pH 9). Preferably ethylenediaminetetramethylenephosphate (EDTMP), diethylenetriamine-pentamethylenephosphonate (DTPMP) and their higher homologs may be considered as the aminoalkanephosphonates. They are preferably used in the form of the neutral-reacting sodium salts, e.g., as the hexasodium salt of EDTMP and/or as the heptasodium and octasodium salts of DTPMP. From the phosphonate class, HEDP is preferably used as a builder. The aminoalkane-phosphonates also have a strong heavy-metal-binding capacity. Accordingly, it may be preferable to use aminoalkanephosphonates, in particular DTPMP, or mixtures of said phosphonates, in particular when the agents also contain bleach.

In addition, all compounds that are capable of forming complexes with alkaline earth ions may be used as cobuilders.

Builder substances may optionally be present in the inventive detergents or cleaning agents in amounts up to 90 wt %. They are preferably present in amounts up to 75 wt %. Inventive detergents have builder contents of 5 wt % to 50 wt % in particular. In inventive agents for cleaning hard surfaces, in particular for machine cleaning of tableware, the builder substance content is 5 wt % to 88 wt % in particular, but preferably no water-insoluble builder materials are used in such agents. In a preferred embodiment of inventive agents for machine washing of tableware in particular, 20 wt % to 40 wt % water-soluble organic builders, in particular alkali citrate, 5 wt % to 15 wt % alkali carbonate and 20 wt % to 40 wt % alkali disilicate are present.

Solvents that may be used in the liquid to gelatinous compositions of detergents and cleaning agents originate from the group of monovalent or polyvalent alcohols, alkanolamines or glycol ethers, for example, if they are miscible with water in the concentration range given. The solvents are preferably selected from ethanol, n-propanol or isopropanol, butanols, ethylene glycol methyl ether, ethylene glycol ethyl ether, ethylene glycol propyl ether, ethylene glycol mono-n-butyl ether, diethylene glycol methyl ether, diethylene glycol ethyl ether, propylene glycol methyl ether, propylene glycol ethyl ether or propylene glycol propyl ether, methoxy-, ethoxy- or butoxytriglycol, 1-butoxyethoxy-2-propanol, 3-methyl-3-methoxybutanol, propylene glycol-t-butyl ether as well as mixtures of these solvents.

Solvents may be used in the inventive liquid to gelatinous detergents and cleaning agents in amounts between 0.1 and 20 wt %, but preferably less than 15 wt % and in particular less than 10 wt %.

To adjust the viscosity, one or more thickeners and/or thickening systems may be added to the inventive composition. These high-molecular substances, which are also known as swelling agents, mostly absorb the liquids and swell in the process, ultimately becoming viscous colloidal solutions or true solutions.

Suitable thickeners are inorganic or polymeric organic compounds. The inorganic thickeners include, for example, polysilicic acids, clay minerals such as montmorillonites, zeolites, silicic acids and bentonites. The organic thickeners come from the groups of natural polymers, the modified natural polymers and the fully synthetic polymers. Such naturally occurring polymers include, for example, agar, carrageenan, gum tragacanth, gum arabic, alginates, pectins, polyoses, guar powder, carob bean powder, starch, dextrins, gelatins and casein. Modified natural substances that are used as thickeners originate mainly from the group of modified starches and celluloses. Examples here include carboxymethylcellulose and other cellulose ethers, hydroxyethyl-cellulose and hydroxypropylcellulose as well as kernel meal ether. Fully synthetic thickeners are polymers such as polyacryl compounds and polymethacryl compounds, vinyl polymers, polycarboxylic acids, polyethers, polyimines, polyamides and polyurethanes.

The thickeners may be present in an amount up to 5 wt %, preferably from 0.05 to 2 wt % and especially preferably from 0.1 to 1.5 wt %, based on the finished composition.

The inventive detergents and cleaning agents may optionally contain as additional conventional ingredients sequestering agents, electrolytes and additional additives such as optical brighteners, graying inhibitors, silver corrosion inhibitors, dye transfer inhibitors, foam inhibitors, abrasives, dyes and/or perfumes as well as microbial active ingredients, UV absorbers and/or enzyme stabilizers.

Inventive textile detergents may contain as optical brighteners derivatives of diaminostilbenedisulfonic acid and/or its alkali metal salts. Suitable examples include salts of 4,4′-bis(2-anilino-4-morpholino-1,3,5-triazinyl-6-amino)stilbene-2,2′-disulfonic acid or compounds having a similar structure and containing a diethanolamino group, a methylamino group, an anilino group or a 2-methoxyethylamino group instead of the morpholino group. In addition, brighteners of the substituted diphenylstyryl type may also be present, e.g., the alkali salts of 4,4′-bis(2-sulfostyryl)diphenyl, 4,4′-bis(4-chloro-3-sulfostyryl)diphenyl or 4-(4-chlorostyryl)-4′-(2-sulfostyryl)diphenyl. Mixtures of the aforementioned optical brighteners may also be used.

Graying inhibitors have the task of keeping the dirt released from the textile fibers suspended in the solution. Water-soluble colloids, usually of an organic nature, are suitable for this purpose, e.g., starch, glue, gelatins, salts of ether carboxylic acids or ether sulfonic acids of starch or cellulose or salts of acidic sulfuric acid esters of cellulose or of starch. Water-soluble polyamides containing acid groups are also suitable for this purpose. In addition, starch derivatives other than those listed above may also be used, e.g., aldehyde starches. Cellulose ethers, e.g., carboxymethylcellulose (Na salt), methylcellulose, hydroxyalkylcellulose and mixed ethers, e.g., methyl-hydroxyethylcellulose, methylhydroxypropylcellulose, methylcarboxymethyl-cellulose and mixtures thereof, e.g., in amounts of 0.1 to 5 wt %, based on the agents, are preferred for use.

To protect silver from corrosion, silver corrosion inhibitors may be used in the inventive cleaning agents for tableware. Such inhibitors are known from the prior art, e.g., benzotriazoles, iron (III) chloride or CoSO₄. As is known from European Patent EP 0 736 084 B1, for example, especially suitable silver corrosion inhibitors for joint use with enzymes include manganese, titanium, zirconium, hafnium, vanadium, cobalt or cerium salts and/or complexes in which said metals are present in one of the oxidation states II, III, IV, V or VI. Examples of such compounds include MnSO₄, V₂O₅, V₂O₄, VO₂, TiOSO₄, K₂TiF₆, K₂ZrF₆, Co(NO₃)₂, Co(NO₃)₃ as well as mixtures thereof.

Soil-release active ingredients or soil repellents are usually polymers which impart dirt-repellent properties to the laundered fiber when used in a detergent and/or which support the dirt release capacity of the other detergent ingredients. A comparable effect may also be observed when they are used in cleaning agents for hard surfaces.

Soil-release active ingredients that are especially effective and have been known for a long time are the copolyesters with dicarboxylic acid units, alkylene glycol units and polyalkylene glycol units. Examples include copolymers or polymer mixtures of polyethylene terephthalate and polyoxyethylene glycol (DT 16 17 141 and/or DT 22 00 911). Unexamined German patent application DT 22 53 063 mentions acidic agents containing, among other things, a copolymer of a dibasic carboxylic acid and an alkylene or cycloalkylene polyglycol. Polymers of ethylene terephthalate and polyethylene oxide terephthalate and their use in detergents are described in the documents DE 28 57 292 and DE 33 24 258 and in European Patent EP 0 253 567. European Patent EP 066 944 relates to agents containing a copolyester of ethylene glycol, polyethylene glycol, aromatic dicarboxylic acids and sulfonated aromatic dicarboxylic acid in certain molar ratios. European Patent EP 0 185 427 discloses methyl or ethyl end-group-capped polyesters with ethylene and/or propylene terephthalate and polyethylene oxide terephthalate units and detergents containing such a soil-release polymer. European Patent EP 0 241 984 relates to a polyester which also contains substituted ethylene units and glycol units in addition to oxyethylene groups and terephthalic acid units. European Patent EP 0 241 985 describes polyesters that contain in addition oxyethylene groups and terephthalic acid units, 1,2-propylene groups, 1,2-butylene groups and/or 3-methoxy-1,2-propylene groups as well as glycerol units and are end-group-capped with C₁ to C₄ alkyl groups. European Patent Application EP 0 272 033 discloses polyesters at least proportionally end-group-capped by C₁₋₄ alkyl or acryl radicals and having polypropylene terephthalate units and polyoxyethylene terephthalate units. European Patent EP 0 274 907 describes sulfoethyl end-group-capped terephthalate-containing soil-release polyesters. According to European Patent Application EP 0 357 280, soil-release polyesters with terephthalate units, alkylene glycol units and poly-C₂₋₄-glycol units are produced by sulfonation of unsaturated end groups. International Patent Application WO 95/32232 relates to acidic, aromatic soil-release-enabling polyesters. International Patent Application WO 97/31085 discloses nonpolymeric soil-repellent active ingredients for materials from cotton with multiple functional units: a first unit, which may be cationic, for example, is capable of adsorption onto the cotton surface through electrostatic interaction, and a second unit, which is hydrophobic, is responsible for the active ingredient remaining at the water/cotton interface.

The dye transfer inhibitors that may be considered for use in the inventive textile detergents include in particular polyvinylpyrrolidones, polyvinylimidazoles, polymeric N-oxides such as poly(vinylpyridine N-oxide) and copolymers of vinylpyrrolidone with vinylimidazole.

When used in machine cleaning methods, it may be advantageous to add foam inhibitors to the respective agents. Suitable foam inhibitors include, for example, soaps of natural or synthetic origin containing a large amount of C₁₈-C₂₄ fatty acids. Suitable nonsurfactant foam inhibitors include, for example, organopolysiloxanes and mixtures thereof with microfine silicic acid, optionally silanized silicic acid as well as paraffins, waxes, microcrystalline waxes or mixtures thereof with silanized silicic acid or bistearylethylenediamide. Mixtures of different foam inhibitors may also be used to advantage, e.g., mixtures of silicones, paraffins or waxes. The foam inhibitors, in particular the foam inhibitors containing silicone and/or paraffin, are preferably bound to a granular, water-soluble and/or dispersible carrier substance. In particular, mixtures of paraffins and bistearylethylenediamides are preferred.

An inventive cleaning agent for hard surfaces may also contain abrasive components, in particular form the group comprising powdered quartz, sawdust, powdered plastics, chalks and glass microbeads as well as mixtures thereof. Abrasives are contained in the inventive cleaning agents preferably in an amount of no more than 20 wt %, in particular in an amount of 5 wt % to 15 wt %.

Dyes and perfumes are added to detergents and cleaning agents to improve the esthetic impression of the products and to make available to the consumer a visually and sensorially “typical and unmistakable” product in addition to the washing and cleaning performance. Perfume oils and/or scents that may be used include individual perfume compounds, e.g., the synthetic products of the type of esters, ethers, aldehydes, ketones, alcohols and hydrocarbons. Perfume compounds of the ester type include, for example, benzyl acetate, phenoxyethyl isobutyrate, p-tert-butylcyclohexyl acetate, linalyl acetate, dimethylbenzylcarbinyl acetate, phenylethyl acetate, linalyl benzoate, benzyl formate, ethylmethylphenyl glycinate, allylcyclohexyl propionate, styrallyl propionate and benzyl salicylate. The ethers include, for example, benzylethyl ether; the aldehydes include, for example, the linear alkanols with 8 to 18 carbon atoms, citral, citronellal, citronellyloxyacetaldehyde, cyclamen aldehyde, hydroxycitronellal, lilial and bourgeonal; the ketones include, for example, the ionones, α-isomethylionone and methyl cedryl ketone; the alcohols include anethol, citronellel, eugenol, geraniol, linalool, phenylethyl alcohol and terpineol; the hydrocarbons include mainly the terpenes, such as limonene and pinene. However, mixtures of different perfumes, which together produce an appealing scent, are preferred. Such perfume oils may also be present as natural perfume mixtures, such as those accessible from plant sources, e.g., pine oil, citrus oil, jasmine oil, patchouli oil, rose oil or ylang-ylang oil. Also suitable are muscatel, sage oil, chamomile oil, clove oil, lemon balm oil, mint oil, cinnamon oil, linden blossom oil, juniper oil, vetiver oil, frankincense oil, galbanum oil and labdanum oil as well as orange blossom oil, neroli oil, orange peel oil and sandalwood oil. The amount of dyes in detergents and cleaning agents is usually less than 0.1 wt %, whereas scents may constitute up to 2 wt % of the total formulation.

The scents may be incorporated directly into the detergents or cleaning agents but it may also be advantageous to apply the scents to carriers which enhance the adherence of the perfume to the material to be cleaned and ensure that the scent is released more slowly for a long-lasting scent, in particular with the treated textiles. Such carrier materials have proven to be, for example, cyclodextrins, where the cyclodextrin-perfume complexes may additionally be coated with other additives. Another preferred carrier for scents is the zeolite X described above, which may also absorb scents instead of or in mixture with surfactants. Therefore, detergents and cleaning agents which contain zeolite X described above and scents that are preferably at least partially absorbed on the zeolite are preferred.

Preferred dyes, selection of which does not pose any problem for those skilled in the art, have a great stability in storage and are insensitive to the other ingredients of the agents and with respect to light as well as not having any pronounced substantivity with respect to textile fibers so as not to stain them.

To combat microorganisms, detergents or cleaning agents may contain antimicrobial active ingredients. Bacteriostatics and bactericides, fungistatics and fungicides, etc. are differentiated here according to the antimicrobial spectrum and mechanism of action. Important substances from these groups include, for example, benzalkonium chlorides, alkylarylsulfonates, halophenols and phenol mercuriacetate. The terms “antimicrobial effect” and “antimicrobial active ingredient” have the usual technical meaning within the context of the inventive teaching, as explained, for example, by K. H. Wallhäuser in “Praxis der Sterilisation, Desinfektion—Konservierung: Keimidentifizierung—Betriebshygiene” [Practice of Sterilization, Disinfection—Preservation: Identi-fication of Microbes—Plant Hygiene] (5^{th} edition, Stuttgart, New York, Thieme, 1995), whereby all the substances having an antimicrobial effect described there may be used. Suitable antimicrobial active ingredients are preferably selected from the groups of alcohols, amines, aldehydes, antimicrobial acids and/or the salts thereof, carboxylic acid esters, acid amides, phenols, phenol derivatives, diphenyls, diphenylalkanes, urea derivatives, oxygen acetals, nitrogen acetals and formals, benzamidines, isothiazolines, phthalimide derivatives, pyridine derivatives, antimicrobial surfactant compounds, guanidines, antimicrobial amphoteric compounds, quinolines, 1,2-dibromo-2,4-dicyanobutane, iodo-2-propylbutylcarbamate, iodine, iodophors, peroxo compounds, halogen compounds and any mixtures of the above.

The antimicrobial active ingredient may be selected from ethanol, n-propanol, isopropanol, 1,3-butandiol, phenoxyethanol, 1,2-propylene glycol, glycerol, undecylenic acid, benzoic acid, salicylic acid, dihydracetic acid, o-phenylphenol, N-methylmorpholinium acetonitrile (MMA), 2-benzyl-4-chlorophenol, 2,2′-methylene-bis-(6-bromo-4-chlorophenol), 4,4′-dichloro-2′-hydroxydiphenylether (dichlosan), 2,4,4′-trichloro-2′-hydroxydiphenyl ether (trichlosan), chlorohexidine, N-(4-chlorophenyl)-N-(3,4-dichlorphenyl)urea, N,N′-(1,10-decane-diyldi-1-pyridinyl-4-ylidene)-bis-(1-octanamine) dihydrochloride, N,N′-bis(4-chlorophenyl)-3,12-diimino-2,4,11,13-tetraazatetradecanediimideamide, glucoprotamines, antimicrobial surfactant quaternary compounds, guanidines including the biguanidines and polyguanidines such as, for example, 1,6-bis-(2-ethylhexylbiguanidohexane) dihydrochloride, 1,6-di-(N₁,N₁′-phenyldiguanido-N₅,N₅′)hexane tetrahydrochloride, 1,6-di-(N₁,N₁′-phenyl-N₁,N₁-methyldiguanido-N₅,N₅′)hexane dihydrochloride, 1,6-di-(N₁,N₁′-o-chlorophenyldiguanido-N₅,N₅′)hexane dihydrochloride, 1,6-di-(N₁,N₁′-2,6-dichlorophenyldiguanido-N₅,N₅′)hexane dihydrochloride, 1,6-di-[N₁,N₁′-β-(p-methoxyphenyl)diguanido-N₅,N₅′]hexane dihydrochloride, 1,6-di-(N₁,N₁′-α-methyl-β-phenyldiguanido-N₅,N₅′)hexane dihydrochloride, 1,6-di-(N₁,N₁′-p-nitrophenyldiguanido-N₅,N₅′)hexane dihydrochloride, ω:ω di-(N₁,N₁′-phenyldiguanido-N₅,N₅′)-di-n-propyl ether dihydrochloride, ω:ω′-di-(N₁,N₁′-p-chlorophenyldiguanido-N₅,N₅′)-di-n-propyl ether tetrahydrochloride, 1,6-di-(N₁,N₁′-2,4-dichlorophenyldiguanido-N₅,N₅′)hexane tetrahydrochloride, 1,6-di-(N₁,N₁′-p-methylphenyldiguanido-N₅,N₅′)hexane dihydrochloride, 1,6-di-(N₁,N′-2,4,5-trichlorophenyldiguanido-N₅,N₅′)hexane tetrahydrochloride, 1,6-di-[N₁,N₁′-α-(p-chlorophenyl)ethyldiguanido-N₅,N₅′]hexane dihydrochloride, ω:ω-di-(N₁,N₁′-p-chlorophenyldiguanido-N₅,N₅′)-m-xylene dihydrochloride, 1,12-di-(N₁,N₁′-p-chlorophenyldiguanido-N₅,N₅′)dodecane dihydrochloride, 1,10-di-(N₁,N₁′-phenyldiguanido-N₅,N₅′)decane tetrahydrochloride, 1,12-di-(N₁,N₁′-phenyldiguanido-N₅,N₅′)dodecane tetrahydrochloride, 1,6-di-(N₁,N₁′-o-chlorophenyldiguanido-N₅,N₅′)hexane dihydrochloride, 1,6-di-(N₁,N₁′-o-chlorophenyldiguanido-N₅,N₅′)hexane tetrahydrochloride, ethylene-bis-(1-tolylbiguanide), ethylene-bis-(p-tolylbiguanide), ethylene-bis-(3,5-dimethylphenylbiguanide), ethylene-bis-(p-tert-amylphenylbiguanide), ethylene-bis-(nonylphenylbiguanide), ethylene-bis-(phenylbiguanide), ethylene-bis-(n-butylphenylbiguanide), ethylene-bis-(2,5-diethoxyphenylbiguanide), ethylene-bis-(2,4-dimethylphenylbiguanide), ethylene-bis-(o-diphenylbiguanide), ethylene-bis-(mixed amyl naphthylbiguanide), N-butylethylene-bis-(phenylbiguanide), trimethylene-bis-(o-tolylbiguanide), N-butyltrimethylene-bis-(phenylbiguanide) and the corresponding salts such as acetates, gluconates, hydrochlorides, hydrobromides, citrates, bisulfites, fluorides, polymaleates, N-cocoalkylsarcosinates, phosphites, hypophosphites, perfluorooctanoates, silicates, sorbates, salicylates, maleates, tartrates, fumarates, ethylenediaminetetraacetates, iminodiacetates, cinnamates, thiocyanates, arginates, pyromellitates, tetracarboxybutyrates, benzoates, glutarates, monofluorophosphates, perfluoropropionates and any mixtures thereof. Also suitable are halogenated xylene and cresol derivatives such as p-chlorometacresol or p-chlorometaxylene as well as natural antimicrobial active ingredients of plant origin (for example, from spices or herbs), animal and microbial origin. Antimicrobially active surfactant quaternary compounds, a natural antimicrobial active ingredient of plant origin and/or a natural antimicrobial active ingredient of animal origin may be preferred; extremely preferred or at least one natural antimicrobial active ingredient of plant origin from the group comprising caffeine, theobromine and theophylline as well as essential oils such as eugenol, thymol and geraniol and/or at least one natural antimicrobial active ingredient of animal origin from the group comprising enzymes such as protein from milk, lysozyme and lactoperoxidase and/or at least one antimicrobially active surfactant quaternary compound with an ammonium group, a sulfonium, a phosphonium group, an iodonium group or an arsonium group, peroxo compounds and chloro compounds may be used. Substances of microbial origin, so-called bacteriocines, may also be used.

The quaternary ammonium compounds (QAC) suitable as antimicrobial active ingredients have the general formula (R¹)(R²)(R³)(R⁴)N⁺X⁻ in which R¹ to R⁴ denote the same or different C₁-C₂₂ alkyl radicals, C₇-C₂₈ aralkyl radicals or heterocyclic radicals, whereby two radicals or, in the case of an aromatic bond as in pyridine, even three radicals together with the nitrogen atom form the heterocycle, e.g., a pyridinium compound or an imidazolinium compound and X⁻ denotes halide ions, sulfate ions, hydroxide ions or similar ions. For an optimal antimicrobial effect, at least one of the radicals preferably has a chain length of 8 to 18 carbon atoms, in particular 12 to 16 carbon atoms.

QACs can be produced by reaction of tertiary amines with alkylating agents, for example, methyl chloride, benzyl chloride, dimethyl sulfate, dodecyl bromide but also ethylene oxide. Alkylation of tertiary amines with a long alkyl radical and two methyl groups succeeds especially easily; quaternation of tertiary mines with two long radicals and one methyl group may also be performed with the help of methyl chloride under mild conditions. Amines having three long alkyl radicals or hydroxy-substituted alkyl radicals are less reactive and are preferably quaternated with dimethyl sulfate.

Suitable QACs include, for example, benzalkonium chloride (N-alkyl-N,N-dimethylbenzylammonium chloride, CAS no. 8001-54-5), benzalkone B (m,p-dichlorobenzyldimethyl-C₁₂-alkylammonium chloride, CAS no. 58390-78-6), benzoxonium chloride (benzyldodecyl-bis-(2-hydroxyethyl)ammonium chloride), cetrimonium bromide (N-hexadecyl-N,N-trimethylammonium bromide, CAS no. 57-09-0), benzethonium chloride (N,N-dimethyl-N-[2-[2-[p-(1,1,3,3-tetramethylbutyl)phenoxy]ethoxy]ethyl]benzylammonium chloride, CAS no. 121-54-0), dialkyldimethylammonium chloride such as di-n-decyldimethylammonium chloride (CAS no. 7173-51-5-5), didecyldimethylammonium bromide (CAS no. 2390-68-3), dioctyidimethylammonium chloride, 1-cetyl-pyridinium chloride (CAS no. 123-03-5) and thiazoline iodide (CAS no. 15764-48-1) as well as mixtures thereof. Especially preferred QACs are the benzalkonium chlorides with C₈-C₁₈ alkyl radicals, in particular C₁₂-C₁₄ alkylbenzyldimethylammonium chloride.

Benzalkonium halides and/or substituted benzalkonium halides are commercially available, for example, from Lonza as Barquat®, from Mason as Marquat®, from Witco/Sherex as Variquat® and from Lonza as Hyamine® as well as from Lonza as Bardac®. Other commercially available antimicrobial active ingredients include N-(3-chloroallyl)hexaminium chloride such as Dowicide® and Dowicil® from Dow, benzethonium chloride such as Hyamine® 1622 from Rohm & Haas, methylbenzethonium chloride such as Hyamine® 10× from Rohm & Haas, cetylpyridinium chloride such as cepacol chloride from Merrell Labs.

The antimicrobial active ingredients are used in amounts of 0.001 wt % to 1 wt %, preferably from 0.001 wt % to 0.8 wt %, especially preferably from 0.005 wt % to 0.3 wt % and in particular from 0.01 to 0.2 wt %.

The inventive detergents or cleaning agents may contain UV absorbers which are absorbed onto the treated textiles and improve the lightfastness of the fibers and/or the lightfastness of other ingredients of the recipe. UV absorbers are understood to organic substances (light protection filters) which are capable of absorbing ultraviolet rays and emitting the energy thereby absorbed in the form of longer-wavelength radiation, e.g., heat.

Compounds which have these desired properties are, for example, the compounds and derivatives of benzophenone with substituents in positions 2 and/or 4 that are active by radiationless deactivation. In addition, substituted benzotriazoles, acrylates with a phenyl substituent in position 3 (cinnamic acid derivatives, optionally with cyano groups in position 2), salicylates, organic nickel complexes and natural substances such as umbelliferone and endogenous urocanic acid. Biphenyl derivatives and especially stilbene derivatives such as those described in EP 0728749 A, for example, and available commercially as Tinosorb® FD or Tinosorb® FR from Ciba have gained special importance. Examples of UV-B absorbers include: 3-benzylidenecamphor and/or 3-benzylidenenorcamphor and derivatives thereof, e.g., 3-(4-methylbenzylidene)camphor, as described in EP 0693471 B1; 4-aminobenzoic acid derivatives, preferably 4-(dimethylamino)benzoic acid 2-ethylhexyl ester, 4-(dimethylamino)benzoic acid 2-octyl ester and 4-(dimethylamino)benzoic acid amyl ester; esters of cinnamic acid, preferably 4-methoxycinnamic acid-2-ethylhexyl ester, 4-methoxycinnamic acid propyl ester, 4-methoxycinnamic acid isoamyl ester, 2-cyano-3,3-phenylcinnamic acid 2-ethylhexyl ester (octocrylene); esters of salicylic acid, preferably salicylic acid 2-ethylhexyl ester, salicylic acid 4-isopropylbenzyl ester, salicylic acid homomethyl ester; derivatives of benzophenone, preferably 2-hydroxy-4-methoxybenzophenone, 2-hydroxy-4-methoxy-4′-methylbenzophenone, 2,2′-dihydroxy-4-methoxybenzohenone; esters of benzalmalonic acid, preferably 4-methoxybenzmalonic acid di-2-ethylhexyl ester; triazine derivatives, e.g., 2,4,6-trianilino-(p-carbo-2′-ethyl-1′-hexyloxy)-1,3,5-triazine and octyltriazone, as described in EP 0818450 A1 or dioctylbutamidotriazone (Uvasorb® HEB); propane-1,3-diones, e.g., 1-(4-tert-butylphenyl)-3-(4′-methoxyphenyl)propane-1,3-dione; ketotricyclo-(5.2.1.0)-decane derivatives, as described in EP 0694521 B1. Also suitable are 2-phenylbenzimidazole-5-sulfonic acid and its alkali salts, alkaline earth salts, ammonium salts, alkylammonium salts, alkanolammonium salts and glucammonium salts; sulfonic acid derivatives of benzophenones, preferably 2-hydroxy-4-methoxybenzophenone-5-sulfonic acid and its salts; sulfonic acid derivatives of 3-benzylidenecamphor, e.g., 4-(2-oxo-3-bornylidenemethyl)benzenesulfonic acid and 2-methyl-5-(2-oxo-3-bornylidene)sulfonic acid and their salts.

Typical UV-A filters include in particular the derivatives of benzoyl methane such as 1-(4′-tert-butylphenyl)-3-(4′-methoxyphenyl)propane-1,3-dione, 4-tert-butyl-4′-methoxydibenzoylmethane (Parsol 1789), 1-phenyl-3-(4′-isopropylphenyl)propane-1,3-dione as well as enamine compounds, as described in DE 19712033 A1 (BASF). The UV-A and UV-B filters may of course also be used in mixtures. In addition to the aforementioned soluble substances, insoluble light protectant pigments, namely finely dispersed, preferably nanoized metal oxides and/or salts may also be used for this purpose. Examples of suitable metal oxides include in particular zinc oxide and titanium dioxide plus the oxides or iron, zirconium, silicon, manganese, aluminum and cerium as well as mixtures thereof. Salts that may be used include silicates (talc), barium sulfate or zinc stearate. The oxides and salts are already being used in the form of pigments for skin care and skin protective emulsions and decorative cosmetics. The particles should have an average diameter of less than 100 nm, preferably between 5 and 50 nm and in particular between 15 and 30 nm. They may have a spherical shape, but particles having an ellipsoidal shape or a shape that otherwise deviates from the spherical shape may also be used. The pigments may also be surface treated, i.e., hydrophilized or hydrophobicized. Typical examples include sheathed titanium dioxides, e.g., titanium dioxide T 805 (Degussa) or Eusolex® T2000 (Merck), preferably silicones and especially preferably trialkoxyoctylsilanes or simethicones may be used as hydrophobic coating agents for this purpose. Micronized zinc oxide is preferably used. Other suitable UV light protectant filters can be found in the review by P. Finkel in SOFW Journal 122 (1996), p. 543.

The UV absorbers are usually used in amounts of 0.01 wt % to 5 wt %, preferably from 0.03 wt % to 1 wt %.

Inventive agents may contain additional enzymes to increase the detergent performance and/or cleaning performance in addition to the inventive proteins, whereby in principle all enzymes established in the prior art may be used for this purpose. These include in particular other proteases, amylases, lipases, hemicellulases, cellulases or oxidoreductases, as well as preferably mixtures thereof. These enzymes are in principle of natural origin; starting from the natural molecules, improved variants that are preferred for use accordingly are available for use in detergents and cleaning agents. Inventive agents contain these additional enzymes, preferably in total amounts of 1×10⁻⁶ to 5 wt %, based on active protein.

Of the other proteases, those of the subtilisin type are preferred. Examples of these include subtilisins BPN′ and Carlsberg, protease BP92, subtilisins 147 and 309, the alkaline protease from Bacillus lentus, subtilisin DY and the enzymes that are to be allocated to the subtilases but no longer belong to the subtilisins in the narrower sense, namely thermitase, proteinase K and the proteases TW3 and TW7. Subtilisin Carlsberg is available in a further developed form under the brand names Alcalase® from the company Novozymes A/S, Bagsvaerd, Denmark. The subtilisins 147 and 309 are distributed under the brand names Esperase® and/or Savinase® by the company Novozymes. Variants carried under the brand name BLAP® and described in particular WO 92/21760 A1, WO 95/23221 A1, WO 02/088340 A2 and WO 03/038082 A2 are derived from the protease from Bacillus lentus DSM 5483 (WO 91/02792 A1). Other usable proteases from various Bacillus sp. and B. gibsonii strains are to be found in the patent applications WO 03/054185, WO 03/056017, WO 03/055974 and WO 03/054184.

Other usable proteases include, for example, the enzymes available under the brand names Durazym®, Relase®, Everlase®, Nafizym, Natalase®, Kannase® and Ovozymes® from the company Novozymes, those available under the brand names Purafect®, Purafect® OxP and Properase® from the company Genencor, the enzyme available under the brand name Protosol® from the company Advanced Biochemicals Ltd., Thane, India, the enzyme available under the brand name Wuxi® from the company Wuxi Snyder Bioproducts Ltd., China, the enzymes available under the brand names Proleather® and Protease P® from the company Amano Pharmaceuticals Ltd., Nagoya, Japan and the enzyme available under the brand name Proteinase K-16 from the company Kao Corp., Tokyo, Japan.

Examples of amylases that may be used according to the invention include the α-amylases from Bacillus licheniformis, from B. amyloliquefaciens or from B. stearothermophilus as well as their further developments that have been improved for use in detergents and cleaning agents. The enzyme from B. licheniformis is available from the company Novozymes under the brand name Termamyl® and from the company Genencor under the brand name Purastar® ST. Further development products of these α-amylases are available from the company Novozymes under the brand names Duramyl® and Termamyl® ultra, from the company Genencor under the brand name Purastar® OxAm and from the company Daiwa Seiko Inc., Tokyo, Japan as Keistase®. The α-amylase from B. amyloliquefaciens is distributed by the company Novozymes under the name BAN® and derived variants of α-amylase from B. stearothermophilus are distributed under the brand names BSG® and Novamyl®, also by the company Novozymes. Other commercial products that may be used include, for example, Amylase LT® and Stainzyme®, the latter also from the company Novozymes.

In addition, the α-amylase from Bacillus sp. A 7-7 (DSM 12368) disclosed in the patent application WO 02/10356 A2 and the cyclodextrin glucanotransferase (CGTase) from B. agaradherens (DSM 9948) described in the patent application WO 02/44350 A2 should also be pointed out for this purpose. Furthermore, the amylolytic enzymes belonging to the sequence space of α-amylases, which is defined in the patent application WO 03/002711 A2, and those described in the patent application WO 03/054177 A2 may also be used. Likewise, fusion proteins of said molecules may also be used, e.g., those known from the patent application DE 10138753 A1.

Furthermore, the further developments of the α-amylase from Aspergillus niger and A. oryzae available from the company Novozymes under the brand name Fungamyl® are also suitable. Another commercial product is Amylase LT®, for example.

Inventive agents may contain lipases or cutinases, in particular because of their triglyceride cleaving activities, but also to create peracids in situ from suitable precursors. These include, for example, the lipases available from Humicola lanuginosa (Thermomyces lanuginosus) and/or lipases that have been developed further, in particular those with the amino acid exchange D96L. They are distributed by the company Novozymes under the brand names Lipolase®, Lipolase® Ulra, LipoPrime®, Lipozyme® and Lipex®, for example. In addition, the cutinases, which were originally isolated from Fusarium solani pisi and Humicola insolens, may also be used, for example. Likewise, usable lipases are also available from the company Amano under the brand names Lipase CE®, Lipase P®, Lipase B® and/or Lipase CES®, Lipase AKG®, Bacillus sp. Lipase®, Lipase AP®, Lipase M-AP® and Lipase AML®. From the company Genencor, the lipases and/or cutinases whose starting enzymes were originally isolated from Pseudomonas mendocina and Fusarium solanii may also be used. Other important commercial products that can be mentioned include the preparations M1 Lipase® and Lipomax® originally distributed by the company Gist-Brocades and the enzymes distributed by the company Meito Sangyo K.K., Japan, under the brand names Lipase MY-30®, Lipase OF® and Lipase PLO, also the product Lumafast® from the company Genencor.

Inventive agents may contain cellulases as pure enzymes, as enzyme preparations or in the form of mixtures in which they advantageously supplement the individual components with regard to their various performance aspects, depending on the intended purpose, in particular if they are intended for treatment of textiles. These performance aspects include in particular contributions to the primary washing performance of the agent, to the secondary washing performance of the agent (antiredeposition effect or graying inhibition) and the finish (fabric effect) up to an including having a “stone-washed” effect.

A usable fungal cellulase preparation that is rich in endoglucanase (EG) and/or further developments thereof are offered by the company Novozymes under the brand names Celluzyme®. The products Endolase® and Carezyme®, which are also available from the company Novozymes, are based on the 50 kD EG and the 43 kD EG, respectively, from H. insolens DSM 1800. Other commercial products from this company that may be used here include Cellulsoft® and Renozyme®. The latter is based on the patent application WO 96/29397 A1. Cellulase variants with improved performance are disclosed in the patent application WO 98/12307 A1, for example. Likewise, the cellulases disclosed in the patent application WO 97/14804 A1 may also be used; for example, the 20 kD EG from Melanocarpus available from the company AB Enzymes of Finland under the brand names Ecostone® and Biotouch® may also be used. Other commercial products from the company AB Enzymes include Econase® and Ecopulp®. Other suitable cellulases from Bacillus sp. CBS 670.93 and CBS 669.93 are disclosed in WO 96/34092 A2, whereby the product from Bacillus sp. CBS 670.93 is available under the brand name Puradex® from the company Genencor. Other commercial products from the company include “Genencor detergent cellulase L” and IndiAge® Neutra.

Inventive agents may contain, in addition to the inventive polypeptides, additional enzymes summarized under the term hemicellulases, in particular to remove certain problem soiling. These include, for example, mannanases, xanthan lyases, pectin lyases (=pectinases), pectin esterases, pectate lyases, xyloglucanases (=xylanases) pullulanases and β-glucanases. Suitable mannanases are available, for example, under the brand names Gamanase® and Pektinex AR® from the company Novozymes, under the brand name Rohapec® B1L from the company AB Enzymes and under the brand name Pyrolase® from the company Diversa Corp., San Diego, Calif., USA. A suitable β-glucanase from a B. alcalophilus is disclosed, for example, in the patent application WO 99/06573 A1. The β-glucanase obtained from B. subtilis is available from the company Novozymes under the brand name Cereflo®.

To increase the bleaching effect, inventive detergents and cleaning agents may contain oxidoreductases, e.g., oxidases, oxygenases, catalases, peroxidases such as halo, chloro, bromo, lignin, glucose or manganese peroxidases, dioxygenases or laccases (phenol oxidases, polyphenol oxidases). Suitable commercial products include Denilite® 1 and 2 from the company Novozymes. In addition, preferably organic, especially preferably aromatic compounds that interact with the enzymes are advantageously also added to intensify the activity of the respective oxidoreductases (enhancers) or to ensure the electron flow in the case of extremely different redox potentials between the oxidizing enzymes and the soiling (mediators).

Enzymes additionally used in the inventive agents originate either originally from microorganisms such as the genera Bacillus, Streptomyces, Humicola or Pseudomonas and/or are produced by suitable microorganisms according to known biotechnological methods, e.g., by transgenic expression hosts of the genus Bacillus or filamentary fungi.

The respective enzymes are advantageously purified by established methods, e.g., by precipitation, sedimentation, concentration, filtration of the liquid phases, microfiltration, ultrafiltration, action of chemicals, deodorization or suitable combinations of these steps.

The inventive polypeptides as well as the enzymes additionally used may be added in any form established according to the prior art to the inventive agents. These include, for example, the solid preparations obtained by granulation, extrusion or lyophilization or, in particular in the case of liquid or gelatinous agents, solutions of the enzymes, advantageously as concentrated as possible, with a low water content and/or mixed with stabilizers.

Alternatively, these proteins may be encapsulated for both the solid and liquid dosage forms, e.g., by spray drying or extrusion of the enzyme solution together with a polymer, preferably natural, or in the form of capsules, e.g., those in which the enzymes are enclosed as in a solidified gel or in those of the core-shell type, in which a core containing enzyme is coated with a protective layer that is impermeable for water, air and/or chemicals. Other active ingredients, e.g., stabilizers, emulsifiers, pigments, bleaches or pigments may be applied in addition in added layers. Such capsules are produced by essentially known methods, e.g., by shake granulation or roll granulation or in fluid-bed processes. Such granules advantageously have a low dust content, e.g., due to the application of polymeric film-forming agents, and are stable in storage due to the coating.

In addition, it is also possible to finish two or more enzymes, an inventive polypeptide and another enzyme together, so that a single granule has multiple enzyme activities.

A protein, in particular the inventive polypeptide, contained in an inventive agent, may be protected in particular during storage from damage, for example, inactivation, denaturing or decomposition due to physical influences, oxidation or proteolytic cleavage. In the case of microbial production of the proteins and/or enzymes, inhibition of proteolysis is especially preferred, in particular when the agents also contain proteases. Preferred inventive agents contain stabilizers for this purpose.

One group of stabilizers comprises reversible protease inhibitors. Frequently benzamidine hydrochloride, borax, boric acid, boronic acids or their salts or esters are used for this purpose, including in particular derivatives with aromatic groups, e.g., ortho-, meta- or para-substituted phenylboronic acids, in particular 4-formylphenylboronic acid and/or the salts or esters of the aforementioned compounds. Peptide aldehyde, i.e., oligopeptides with a reduced C terminus, in particular those of 2 to 50 monomers are used for this purpose. The peptidic reversible protease inhibitors include ovomucoid and leupeptin, among others. Specific reversible peptide inhibitors for the protease subtilisin as well as fusion proteins from proteases and specific peptide inhibitors are also suitable for this purpose.

Additional enzyme stabilizers are amino alcohols such as mono-, di-, triethanol- and propanolamine and mixtures thereof, aliphatic carboxylic acids up to C₁₂ such as succinic acid, other dicarboxylic acids or salts of said acids. End-group-capped fatty acid amide alkoxylates are also suitable for this purpose. Certain organic acids used as builders are capable of additionally stabilizing an enzyme contained in the agent as disclosed in WO 97/18287.

Low aliphatic alcohols, but especially polyols, for example, glycerol, ethylene glycol, propylene glycol or sorbitol are other enzyme stabilizers that are frequently used. Diglycerol phosphate also protects against denaturing due to physical influences. Likewise, calcium and/or magnesium salts such as calcium acetate or calcium formate are used.

Polyamide oligomers or polymeric compounds such as lignin, water-soluble vinyl copolymers or cellulose ethers, acrylic polymers and/or polyamides stabilize the enzyme preparation with respect to physical influences or fluctuations in pH, among other things. Polymers containing polyamine N-oxide act as enzyme stabilizers and as dye transfer inhibitors at the same time. Other polymeric stabilizers include linear C₈-C₁₈ polyoxyalkylenes. Alkyl polyglycosides can also stabilize the enzymatic components of the inventive agent and are preferably able to additionally increase their performance. Crosslinked compounds containing nitrogen preferably fulfill a double function as soil-release agents and as enzyme stabilizers. Hydrophobic nonionic polymer stabilizes in particular a cellulase which may optionally also be present.

Reducing agents and antioxidants increase the stability of the enzymes with respect oxidative degradation; for example, reducing agents containing sulfur are customary for this purpose. Other examples include sulfite and reducing sugars.

Combinations of stabilizers, e.g., of polyols, boric acid and/or borax, the combination of boric acid or borate, reducing salts and succinic acid or other dicarboxylic acids or the combination of boric acid or borate with polyols or polyamino compounds and with reducing salts are especially preferred. The effect of peptide-aldehyde stabilizers is advantageously enhanced by the combination with boric acid and/or boric acid derivatives and polyols and even further by the additional effect of divalent cations, e.g., calcium ions.

Since the inventive agents may be offered in all conceivable forms, the inventive polypeptides in all formulations that are expedient for addition to the respective agents constitute the respective embodiments of the present invention. These include, for example, liquid formulations, solid granules or capsules.

The encapsulated form is recommended to protect the enzymes or other ingredients from other components, e.g., bleaching agents or to allow controlled release. Depending on the size of these capsules, a distinction is made according to millicapsules, microcapsules and nanocapsules, microcapsules being especially preferred for enzymes. Such capsules are disclosed, for example, in the patent applications WO 97/24177 and DE 19918267. One possible encapsulation method consists of the fact that the proteins, starting from a mixture of the protein solution with a solution or suspension of starch or a starch derivative, are encapsulated in this substance. Such an encapsulation method is described in the patent application WO 01/38471.

In the case of solid agents, the proteins—inventive polypeptides as well as additional enzymes optionally contained therein—may be used, e.g., in dried, granulated and/or encapsulated form. They may be added separately, i.e., as a separate phase, or together with other ingredients in the same phase with or without compacting. If microencapsulated enzymes are to be processed in solid form, the water may be removed from the aqueous solutions obtained from workup by using methods known from the prior art, e.g., spray drying, centrifugation or resolubilization. The particles obtained in this way usually have a particle size between 50 μm and 200 μm.

Starting from a protein production performed according to the prior art and preparation in concentrated aqueous or nonaqueous solution, suspension or emulsion, but also in gel form or encapsulated or as a dried powder, the proteins may be added to liquid, gelatinous or pasty inventive agents. Such inventive detergents or cleaning agents are usually produced by simple mixing of the ingredients which may be added in substance or as a solution in an automatic mixer.

An inventive cleaning agent, in particular an inventive cleaner for hard surfaces, may also contain one or more propellants (INCI propellants), usually in an amount of 1 to 80 wt %, preferably 1.5 to 30 wt %, in particular 2 to 10 wt %, especially preferably 2.5 to 8 wt %, extremely preferably 3 to 6 wt %.

Propellants are propellant gases that are conventional according to the invention, in particular liquefied or compressed gases. The choice depends on the product to be sprayed and the field of application. When using compressed gases such as nitrogen, carbon dioxide or nitrous oxide, which are generally insoluble in the liquid cleaning agent, the operating pressure drops with each operation of the valve. Liquefied gases (liquid gases) as the propellant, which are soluble in the cleaning agent or which act as a solvent themselves, offer the advantage of a uniform operating pressure and a uniform distribution because the propellant evaporates in air and takes up a volume several hundred times greater.

Thus the following propellants according to the INCI designations are suitable: butane, carbon dioxide, dimethyl carbonate, dimethyl ether, ethane, hydrochlorofluorocarbon 22, hydrochlorofluorocarbon 142b, hydrofluorocarbon 152a, hydrofluorocarbon 134a, hydrofluorocarbon 227ea, isobutane, isopentane, nitrogen, nitrous oxide, pentane, propane. However, chlorofluorocarbons (fluorochlorohydrocarbons, FCHC) as propellants are preferably largely omitted and in particular are completely omitted because of their harmful effect on the ozone shield of the atmosphere, the so-called ozone layer, which protects against hard UV radiation.

Preferred propellants are liquid gases. Liquid gases are gases which can usually be converted from the gaseous state to the liquid state at low pressures and at 20° C. In particular, however, liquid gases are understood to include the hydrocarbons propane, propene, butane, butene, isobutane (2-methylpropane), isobutene (2-methylpropene, isobutylene) and mixtures thereof, which are obtained in oil refineries as byproducts of distillation and cracking of petroleum and in processing of natural gas in separation of gasoline.

The cleaning agent especially preferably contains propane, butane, and/or isobutane, in particular propane and butane, extremely preferably propane butane and isobutane as one or more propellants.

An important task of the enzyme preparation and in particular of the inventive polypeptides is, as stated previously, the primary washing performance. In addition to the primary washing performance, the proteases contained in detergents, however, may also fulfill the function of activating other enzymatic constituents by proteolytic cleavage or inactivating them after a corresponding treatment time. One embodiment of the present invention likewise includes agents having capsules of protease-sensitive material which are hydrolyzed, e.g., by inventive proteins at an intended point in time and release their content. Inventive polypeptides may thus also be used for inactivation reactions, activation reactions or release reactions, in particular in multiphase agents.

Another embodiment of this subject matter of the invention thus also includes accordingly the use of an inventive polypeptide for activation, the activation or release of ingredients of detergents or cleaning agents.

In a preferred embodiment, the agent is designed with an inventive polypeptide so that it may regularly be used as a care agent, e.g., by adding it to the washing process, using it after washing or applying it independently of washing. The desired effect consists of maintaining a smooth surface structure of the textile over a long period of time and/or preventing and/or reducing damage to the fabric.

Methods for machine cleaning of textiles or of hard surfaces in which an inventive polypeptide is used in at least one of the process steps constitute a separate subject matter of the invention.

Of these, methods in which the inventive polypeptide is used in an amount from 40 μg to 4 g, preferably from 50 μg to 3 g, especially preferably from 100 μg to 2 g and most especially preferably from 200 μg to 1 g per application are preferred. This includes all integral and nonintegral values between these numbers.

This include both manual and machine processes, but machine processes are preferred because of their more precise controllability with regard to the amounts used and the treatment times, for example.

Methods of cleaning textiles are characterized in general by the fact that different cleaning-active substances are applied to the material for cleaning in several process steps and then are washed out after the treatment time, or the material for cleaning is otherwise treated with a detergent or a solution of this agent. The same thing applies to methods for cleaning all other materials in addition to textiles which are combined under the heading of hard surfaces. All conceivable washing or cleaning methods may be improved by the inventive proteins in at least one of the process steps and then constitute embodiments of the present invention.

Since preferred inventive polypeptides naturally already have a protein dissolving activity and manifest this even in media that do not otherwise have any cleaning performance, e.g., in plain buffer, a single substep of such a process for machine cleaning of textiles may consist of the fact that, if desired, an inventive polypeptide is applied as the only component with an active cleaning effect in addition to stabilizing compounds, salts or buffer substances. This constitutes an especially preferred embodiment of the present invention.

In another preferred embodiment of such processes, the respective inventive polypeptides are provided within the context of one of the aforementioned recipes for inventive agents, preferably inventive detergents and/or cleaning agents.

A separate subject matter of the invention includes the use of an inventive alkaline protease as described above for cleaning textiles or hard surfaces.

The concentration ranges given above also apply accordingly preferably for these applications.

Inventive proteases may be used in particular according to the properties described above and the methods described above to eliminate protein-based soiling from textiles or from hard surfaces. Embodiments include, for example, hand washing, manual removal of spots from textiles or from hard surfaces or use in conjunction with a machine method.

In a preferred embodiment of this application, the respective inventive alkaline proteases are provided within the context of one of the recipes given above for inventive agents, preferably detergents and/or cleaning agents.

Another subject matter of the present invention is also a product containing an inventive composition and/or an inventive detergent or cleaning agent, in particular an inventive cleaner for hard surfaces and a spray dispenser. The product may be a single chamber container as well as multichamber container, in particular a two chamber container. The spray dispenser here is preferably a manually activated spray dispenser, selected in particular from the group comprising aerosol spray dispenser (pressurized gas containers, also known as spray cans), spray dispensers that automatically build up a pressure, pump spray dispensers and trigger spray dispensers, in particular pump spray dispensers and trigger spray dispensers with a container made of transparent polyethylene or polyethylene terephthalate. Spray dispensers are described in greater detail in WO 96/04940 (Procter & Gamble) and the US Patents cited therein for spray dispensers, to all of which reference is made in this regard and the content of which is herewith included in this patent application. Trigger spray dispensers and pump atomizers have the advantage in comparison with pressurized gas containers that no propellant need be used. The enzyme in this embodiment, optionally even in immobilized form on the particles, may be added to the agent and thereby dosed as a cleaning foam through suitable attachments, nozzles, etc. through which the particles can pass (so-called nozzle valves) on the spray dispenser.

The following examples illustrate the invention further without being limited to them.

### EXEMPLARY EMBODIMENTS

All the working steps of molecular biology conform to standard methods such as those described, for example, in the handbook by Fritsch, Sambrook and Maniatis “Molecular Cloning: A Laboratory Manual”, Cold Spring Harbour Laboratory Press, New York, 1989 or comparable reference works. Enzymes and kits were used according to the instructions of the respective manufacturer.

### Example 1

### Isolation and Identification of a Proteolytically Active Bacterial Strain

0.1 g of a soil sample was suspended in 1 mL sterile NaCl and plated out on agar plates containing powdered milk (1.50% agar, 0.1% K₂NPO₄, 0.5% yeast extract, 1% peptone, 1% powdered milk, 0.02% MgSO₄.7H₂O, 0.40/0 Na₂CO₃, pH 9.6) and incubated at 30° C. A proteolytically active bacterium which was identified by the German Collection of Microorganisms and Cell Cultures (DSMZ) as Bacillus pumilus was isolated on the basis of a clear zone.

TABLE 1
Microbiological properties of the Bacillus pumilus
strain (determination by the DSMZ).

| Property | Result |
|---|---|
| Cell shape | Rods |
| Width (μm) | 0.6-0.8 |
| Length (μm) | 2.0-3.0 |
| Spores | positive, oval |
| Sporangium swollen | negative |
| Catalase | positive |
| Oxidase | positive |
| Anaerobic growth | negative |
| VP reaction | negative |
| pH in VP medium | 6.4 |
| Maximum temperature | |
| Growth positive at ° C. | 50 |
| Growth negative at ° C. | 55 |
| Growth in | |
| Medium pH 5.7 | positive |
| NaCl 2% | positive |
| 5% | positive |
| 7% | positive |
| 10% | weak |
| Acid from (ASS) | |
| D-glucose | positive |
| L-arabinose | positive |
| D-xylose | positive |
| D-mannitol | positive |
| D-fructose | positive |
| Gas from glucose | negative |
| Hydrolysis of | |
| Starch | negative |
| Gelatin | positive |
| Tween 80 | positive |
| Casein | positive |
| Utilization of | |
| Citrate (Koser) | positive |
| Propionate | negative |
| Lysozyme medium | positive |
| Indole reaction | negative |
| Phenylalanine deaminase | negative |
| Arginine dihydrolase | negative |
| Sample of cellular fatty acids | Typical of Bacillus subtilis |
| Partial sequencing of 16 S-rDNA | 99.6% similar with Bacillus pumilus |

### Example 2

### Cloning and Sequencing of Mature Protease

Chromosomal DNA from Bacillus pumilus was prepared according to standard methods, treated with the restriction enzyme Sau 3A, and the resulting fragments were cloned in the vector pAWA22. This is an expression vector derived from pBC16 for use in Bacillus species (Bernhard et al. (1978), J. Bacteriol., vol. 133 (2), pp. 897-903). This vector was transformed into the host strain Bacillus subtilis DB 104 (Kawamura and Doi (1984), J. Bacteriol., vol. 160 (1), pp. 442-444).

The transformants were first regenerated on DM3 medium (8 g/L agar, 0.5M succinic acid, 3.5 g/L K₂HPO₄, 1.5 g/L KH₂PO₄, 20 mM MgCl₂, 5 g/L casiamino acids, 5 g/L yeast extract, 6 g/L glucose, 0.1 g/L BSA) and then transfer-inoculated on TBY skim milk plates (10 g/L peptone, 10 g/L powdered milk (see above), 5 g/L yeast, 5 g/L NaCl, 15 g/L agar). Proteolytically active clones were identified on their basis of the lysis zones. One of the resulting proteolytically active clones was selected, its plasmid was isolated and the insert was sequenced according to standard methods.

The resulting insert contained an open reading frame of approx. 1.2 kb. Its sequence is given in the sequence protocol under the designation SEQ ID NO. 1. It comprises 1152 bp. The amino acid sequence derived from it comprises 383 amino acids, followed by a stop codon. It is given in the sequence protocol under SEQ ID NO. 2. Of these, the first 108 amino acids are presumably not included in the mature protein, thus presumably resulting in a length of 275 amino acids for the mature protein.

These sequences were compared with the protease sequences obtainable from the generally accessible databases Swiss-Prot (Geneva Bioinformatics (GeneBio) S.A., Geneva, Switzerland; http://www.genebio.com/sprot.html) and GenBank (National Center for Biotechnology Information NCBI, National Institutes of Health, Bethesda, Md., USA). The enzymes summarized in Table 2 below were identified as the nearest similar enzymes.

TABLE 2
Homology of the alkaline protease from Bacillus pumilus
with the nearest similar proteins.

| Enzyme | | Ident. k. | Ident. m. | Ident. | Ident. m. |
|---|---|---|---|---|---|
| ID | Organism | DNA | DNA | Propre | Prot. |
| Q2HXI3 | Bacillus pumilus | 91 | 91 | 98 | 98 |
| Q6SIX5 | Bacillus pumilus | 91 | 90 | 97 | 98 |
| Q9KWR4 | Bacillus pumilus | 91 | 90 | 98 | 98 |
| Q5XPN0 | Bacillus pumilus | 94 | 95 | 97 | 97 |

Wherein the meanings are:
ID The registration numbers in the GenBank and Swiss-Prot databases
Ident. k. DNA Identity on a DNA level for the complete DNA in %
Ident. m. DNA Identity on a DNA level for the DNA coding for the mature protein in %
Ident. Propre Identity on an amino acid level, based on the propreprotein in %
Ident. m. Prot. Identity on an amino acid level, based on the mature protein in %
n Not given in the databases.

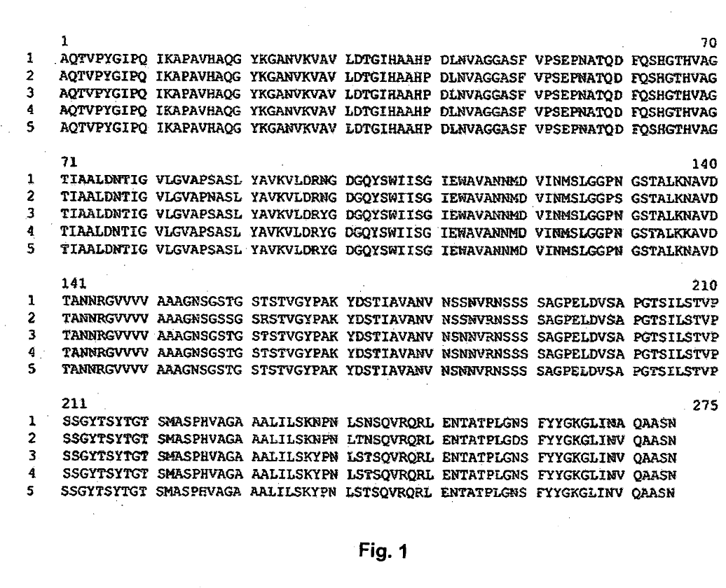

The amino acid sequences of these proteases are also compared with one another in the alignment in FIG. 1.

### Example 3

### SDS Polyacryl Gel Electrophoresis and Isoelectric Focusing

In denaturing SDS polyacryl gel electrophoresis in the PHAST® system of Pharmacia-Amersham Biotech, Sweden, the alkaline protease from Bacillus pumilus obtained according to Examples 2 and 3 has a molecular weight of 27 kD.

According to isoelectric focusing, also in the PHAST® system of Pharmacia-Amersham Biotech, the isoelectric point of the alkaline protease from B. gibsonii is more than 8.5.

### Example 4

### Determining the Washing Performance when Used in Commercial Liquid Detergent

For this example, textiles with standardized soiling were used, having been ordered from the Swiss Materials Testing and Experimental Institute in St. Gallen, Switzerland (EMPA) or the Laundry Research Institute, Krefeld. The following soiling and textiles were used: A (salad dressing on cotton, CFT CS-6), B (grass on cotton, CFT CS-8), C (blood on cotton, EMPA E-111) and D (milk/cocoa on cotton, EMPA E-112). Furthermore, the average was formed over all soilings tested (E).

With this test material, various detergent formulations were tested launderometrically for their washing performance. To do so, a bath ratio of 1:12 was adjusted and the textiles were washed for 30 minutes at a temperature of 30° C. and/or 60° C. The dosage was 4.4 g of the respective agent per liter of wash bath. The water hardness was 160 of [German] water hardness.

A basic detergent formulation of the following composition was used as the control detergent (all values given in wt %): 0.3-0.5% xanthan gum, 0.2-0.4% foam suppressant, 6-7% glycerol, 0.3-0.5% ethanol, 4-7% FAEOS, 24-28% nonionic surfactants, 1% boric acid, 1-2% sodium citrate (dihydrate), 2-4% sodium carbonate, 14-16% coconut fatty acids, 0.5% HEDP, 0-0.4% PVP, 0-0.05% optical brightener, 0-0.001% dye, remainder demineralized water. It was mixed with the following proteases for the various experimental series, so that a final concentration of 5625 PE of proteolytic activity per liter of wash bath was obtained in each case: B. lentus alkaline protease F 49 (WO 95/23221), B. lentus alkaline protease X (WO 92/21760) and/or the inventive protease from Bacillus pumilus.

After washing, the degree of whiteness of the washed textiles was measured. The measured was performed on a Datacolor SF500-2 spectrometer at 460 nm (UV blocking filter 3), 30 mm aperture, without glass, type of light D65, 10°, d/8°. The averages of four measurements each are given. They allow a direct inference regarding the contribution of the enzyme contained in the agent to the washing performance of the agent used.

TABLE 3
Washing results at 30° C.

| Basic detergent | A | B | C | D | E |
|---|---|---|---|---|---|
| Inventive protease from B. pumilus | 55.5 | 73.7 | 53.7 | 54.0 | 59.2 |
| B. lentus alkaline protease F 49 | 50.9 | 69.1 | 44.3 | 49.6 | 53.4 |
| B. lentus alkaline protease X | 50.6 | 69.4 | 45.8 | 50.6 | 54.1 |
| LSD | 1.4 | 1.1 | 2.8 | 2.4 | |

TABLE 4
Washing results at 60° C.

| Basic detergent | A | B | C | D | E |
|---|---|---|---|---|---|
| Inventive protease from B. pumilus | 61.5 | 77.0 | 56.8 | 57.9 | 63.3 |
| B. lentus alkaline protease F 49 | 56.3 | 72.9 | 45.6 | 56.1 | 57.7 |
| B. lentus alkaline protease X | 56.4 | 73.6 | 45.3 | 55.7 | 57.8 |

It can be seen that the inventive protease from B. pumilus exceeds the established proteases B. lentus alkaline protease F 49 and B. lentus alkaline protease X on all the soiling tested and at both temperatures tested.

### Example 5

### Enzymatic Properties

Using the inventive purified alkaline protease from B. pumilus, experiments were conducted with casein at various temperature and various pH levels. It was found that the optimum activity for cleavage of casein was 60° C., whereas the optimum pH for cleavage of casein was 10.5.

1. An isolated polynucleotide comprising a nucleic acid sequence having at least 95% identity with the nucleic acid sequence of SEQ ID NO:1, SEQ ID NO:12, SEQ ID NO:13, or SEQ ID NO:14, or the complement thereof.

2. The polynucleotide of claim 1, wherein the polynucleotide is isolated from a Gram positive bacterium of the genus Bacillus.

3. The polynucleotide of claim 2, wherein the Bacillus is Bacillus pumilus.

4. The polynucleotide of claim 2, wherein the Bacillus is Bacillus pumilus deposited as DSMZ 18097.

5. A vector comprising the polynucleotide of claim 1.

6. The vector of claim 5, wherein the vector is a cloning vector or an expression vector.

7. An isolated protein comprising a polypeptide having at least 98.5% identity with SEQ ID NO:2, SEQ ID NO:8, SEQ ID NO:9, SEQ ID NO:10, or SEQ ID NO:11, wherein the polypeptide has alkaline protease activity.

8. The protein of claim 7, wherein the polypeptide comprises SEQ ID NO:2.

9. The protein of claim 7, wherein the polypeptide is isolated from a Gram positive bacterium of the genus Bacillus.

10. The protein of claim 7, wherein the Bacillus is Bacillus pumilus.

11. The protein of claim 7, wherein the Bacillus is Bacillus pumilus deposited as DSMZ 18097.

12. An isolated cell comprising the vector of claim 5.

13. The cell of claim 12, wherein the cell is capable of expressing a polypeptide having at least 98.5% identity with SEQ ID NO:2, SEQ ID NO:8, SEQ ID NO:9, SEQ ID NO:10, or SEQ ID NO:11, wherein the polypeptide has alkaline protease activity.

14. The cell of claim 12, wherein the cell is a Gram negative bacterium of the genera Escherichia or Klebsiella or a Gram positive bacterium of the genera Bacillus, Staphylococcus, or Corynebacterium.

15. The cell of claim 14, wherein the Bacillus is Bacillus pumilus.

16. The cell of claim 15, wherein the Bacillus is Bacillus pumilus deposited as DSMZ 18097.

17. An isolated alkaline protease encoded by a polynucleotide comprising at least 950% identity with the nucleic acid sequence of SEQ ID NO:1, SEQ ID NO:12, SEQ ID NO:13, or SEQ ID NO:14.

18. A washing or cleaning product comprising a protein comprising a polypeptide having at least 80% identity with SEQ ID NO:2 or SEQ ID NO:10, wherein the polypeptide has alkaline protease activity.

19. The washing or cleaning product of claim 18, comprising the protein in an amount of from about 2 micrograms to about 20 milligrams.

20. The washing or cleaning product of claim 18, further comprising at least one additional protease, amylase, cellulase, hemicellulase, oxidoreductase, or lipase.

21. The washing or cleaning product of claim 18, further comprising at least one additional component selected from the group consisting of surfactants, builders, acids, alkaline substances, hydrotropes, solvents, thickeners, bleaching agents, dyes, perfumes, corrosion inhibitors, sequestering agents, electrolytes, optical brighteners, graying inhibitors, silver corrosion inhibitors, dye transfer inhibitors, foam inhibitors, UV absorbers, solvents, abrasives, antistatics, pearlizing agents and skin protectants.

22. A method for cleaning a textile or surface, comprising contacting the textile or surface with the protein of claim 7.

23. A method for cleaning a textile or surface, comprising contacting the textile or surface with the washing or cleaning product of claim 18.

24. The method of claim 22, wherein a biofilm is present on the textile or surface.

25. The method of claim 23, wherein a biofilm is present on the textile or surface.


SEQUENCE LISTING (14 sequences)

SEQ ID NO: 1 - DNA, 1152 bp, Bacillus pumilus
ttgtgcgtgaaaaagaaaaatgtaatgacaagtgttttattggctgtccctcttctgttttcagcagggtttggaggctccatggcaaatgccgagacggtctcaaagtcagatagtgaaaagagctatattgttggctttaaagcctctgccaccacaaacagctctaagaaacaagccgtcactcaaaatggcggaaaactagaaaagcaatatcgtcttattaatgccgcacaagtaaagatgtccgaacaagccgcaaaaaaacttgaacatgaccctagcattgcttacgtagaagaagaccacaaagcagaagcatatgcacaaaccgtcccttatggaatccctcaaatcaaagctccagctgtacacgctcaaggttataaaggtgctaatgtcaaagtagctgtccttgatactggaatccacgctgcacatcctgacttaaatgttgcaggcggtgccagcttcgtcccttcagagccaaatgccacccaagactttcaatcacatggaactcacgtagccggaaccattgctgcccttgataacacaattggtgttcttggggtcgctccaagtgcttccctatatgctgttaaagtattagaccgcaatggcgacggacaatacagctggatcattagcggtattgaatgggctgtagccaataacatggatgtcatcaatatgagcttaggtggaccaaacggttcaacagcgcttaaaaatgccgttgatacagcaaataaccgcggagtcgttgttgtggcggccgcaggtaattcaggttccactggctctacaagtacagttggctatccagcaaaatatgattctacaattgccgttgccaatgtaaacagcagcaatgtcagaaactcgtcttccagcgcaggtcctgaattagatgtttctgcacctggtacttctattttaagtacagtaccaagcagtggatacacatcttatactggaacttctatggcgtctcctcatgtagcaggagcagcagcgcttattctttctaaaaatccgaacctatcaaattcacaggttcgccagcgcttagaaaacacggcaacaccgcttggtaactccttctattacggaaaagggttaatcaacgctcaagcggcttctaactaa

SEQ ID NO: 2 - protein, 383 aa, Bacillus pumilus
MCVKKKNVMTSVLLAVPLLFSAGFGGSMANAETVSKSDSEKSYIVGFKASATTNSSKKQAVTQNGGKLEKQYRLINAAQVKMSEQAAKKLEHDPSIAYVEEDHKAEAYAQTVPYGIPQIKAPAVHAQGYKGANVKVAVLDTGIHAAHPDLNVAGGASFVPSEPNATQDFQSHGTHVAGTIAALDNTIGVLGVAPSASLYAVKVLDRNGDGQYSWIISGIEWAVANNMDVINMSLGGPNGSTALKNAVDTANNRGVVVVAAAGNSGSTGSTSTVGYPAKYDSTIAVANVNSSNVRNSSSSAGPELDVSAPGTSILSTVPSSGYTSYTGTSMASPHVAGAAALILSKNPNLSNSQVRQRLENTATPLGNSFYYGKGLINAQAASN

SEQ ID NO: 3 - protein, 275 aa, Bacillus pumilus
AQTVPYGIPQIKAPAVHAQGYKGANVKVAVLDTGIHAAHPDLNVAGGASFVPSEPNATQDFQSHGTHVAGTIAALDNTIGVLGVAPSASLYAVKVLDRNGDGQYSWIISGIEWAVANNMDVINMSLGGPNGSTALKNAVDTANNRGVVVVAAAGNSGSTGSTSTVGYPAKYDSTIAVANVNSSNVRNSSSSAGPELDVSAPGTSILSTVPSSGYTSYTGTSMASPHVAGAAALILSKNPNLSNSQVRQRLENTATPLGNSFYYGKGLINAQAASN

SEQ ID NO: 4 - protein, 275 aa, Bacillus pumilus
AQTVPYGIPQIKAPAVHAQGYKGANVKVAVLDTGIHAAHPDLNVAGGASFVPSEPNATQDFQSHGTHVAGTIAALDNTIGVLGVAPNASLYAVKVLDRNGDGQYSWIISGIEWAVANNMDVINMSLGGPSGSTALKNAVDTANNRGVVVVAAAGNSGSSGSRSTVGYPAKYDSTIAVANVNSSNVRNSSSSAGPELDVSAPGTSILSTVPSSGYTSYTGTSMASPHVAGAAALILSKNPNLTNSQVRQRLENTATPLGDSFYYGKGLINVQAASN

SEQ ID NO: 5 - protein, 275 aa, Bacillus pumilus
AQTVPYGIPQIKAPAVHAQGYKGANVKVAVLDTGIHAAHPDLNVAGGASFVPSEPNATQDFQSHGTHVAGTIAALDNTIGVLGVAPSASLYAVKVLDRYGDGQYSWIISGIEWAVANNMDVINMSLGGPNGSTALKNAVDTANNRGVVVVAAAGNSGSTGSTSTVGYPAKYDSTIAVANVNSNNVRNSSSSAGPELDVSAPGTSILSTVPSSGYTSYTGTSMASPHVAGAAALILSKYPNLSTSQVRQRLENTATPLGNSFYYGKGLINVQAASN

SEQ ID NO: 6 - protein, 275 aa, Bacillus pumilus
AQTVPYGIPQIKAPAVHAQGYKGANVKVAVLDTGIHAAHPDLNVAGGASFVPSEPNATQDFQSHGTHVAGTIAALDNTIGVLGVAPSASLYAVKVLDRYGDGQYSWIISGIEWAVANNMDVINMSLGGPNGSTALKKAVDTANNRGVVVVAAAGNSGSTGSTSTVGYPAKYDSTIAVANVNSNNVRNSSSSAGPELDVSAPGTSILSTVPSSGYTSYTGTSMASPHVAGAAALILSKYPNLSTSQVRQRLENTATPLGNSFYYGKGLINVQAASN

SEQ ID NO: 7 - protein, 275 aa, Bacillus pumilus; identical to SEQ ID NO: 5

SEQ ID NO: 8 - protein, 51 aa, Bacillus pumilus
MCVKKKNVMTSVLLAVPLLFSAGFGGSMANAETVSKSDSEKSYIVGFKASA

SEQ ID NO: 9 - protein, 108 aa, Bacillus pumilus
MCVKKKNVMTSVLLAVPLLFSAGFGGSMANAETVSKSDSEKSYIVGFKASATTNSSKKQAVTQNGGKLEKQYRLINAAQVKMSEQAAKKLEHDPSIAYVEEDHKAEAY

SEQ ID NO: 10 - protein, 275 aa, Bacillus pumilus; identical to SEQ ID NO: 3

SEQ ID NO: 11 - protein, 172 aa, Bacillus pumilus
NGDGQYSWIISGIEWAVANNMDVINMSLGGPNGSTALKNAVDTANNRGVVVVAAAGNSGSTGSTSTVGYPAKYDSTIAVANVNSSNVRNSSSSAGPELDVSAPGTSILSTVPSSGYTSYTGTSMASPHVAGAAALILSKNPNLSNSQVRQRLENTATPLGNSFYYGKGLINA

SEQ ID NO: 12 - DNA, 153 bp, Bacillus pumilus
ttgtgcgtgaaaaagaaaaatgtaatgacaagtgttttattggctgtccctcttctgttttcagcagggtttggaggctccatggcaaatgccgagacggtctcaaagtcagatagtgaaaagagctatattgttggctttaaagcctctgcc

SEQ ID NO: 13 - DNA, 324 bp, Bacillus pumilus
ttgtgcgtgaaaaagaaaaatgtaatgacaagtgttttattggctgtccctcttctgttttcagcagggtttggaggctccatggcaaatgccgagacggtctcaaagtcagatagtgaaaagagctatattgttggctttaaagcctctgccaccacaaacagctctaagaaacaagccgtcactcaaaatggcggaaaactagaaaagcaatatcgtcttattaatgccgcacaagtaaagatgtccgaacaagccgcaaaaaaacttgaacatgaccctagcattgcttacgtagaagaagaccacaaagcagaagcatat

SEQ ID NO: 14 - DNA, 828 bp, Bacillus pumilus
gcacaaaccgtcccttatggaatccctcaaatcaaagctccagctgtacacgctcaaggttataaaggtgctaatgtcaaagtagctgtccttgatactggaatccacgctgcacatcctgacttaaatgttgcaggcggtgccagcttcgtcccttcagagccaaatgccacccaagactttcaatcacatggaactcacgtagccggaaccattgctgcccttgataacacaattggtgttcttggggtcgctccaagtgcttccctatatgctgttaaagtattagaccgcaatggcgacggacaatacagctggatcattagcggtattgaatgggctgtagccaataacatggatgtcatcaatatgagcttaggtggaccaaacggttcaacagcgcttaaaaatgccgttgatacagcaaataaccgcggagtcgttgttgtggcggccgcaggtaattcaggttccactggctctacaagtacagttggctatccagcaaaatatgattctacaattgccgttgccaatgtaaacagcagcaatgtcagaaactcgtcttccagcgcaggtcctgaattagatgtttctgcacctggtacttctattttaagtacagtaccaagcagtggatacacatcttatactggaacttctatggcgtctcctcatgtagcaggagcagcagcgcttattctttctaaaaatccgaacctatcaaattcacaggttcgccagcgcttagaaaacacggcaacaccgcttggtaactccttctattacggaaaagggttaatcaacgctcaagcggcttctaactaa